SEQUENCE LISTING (7 sequences)

SEQ ID NO: 1 - DNA, 20 bp, artificial sequence
  synthetic sequence K3 examplary CpG sequence
  phosphorothioate linkage (1)..(19)
atcgactctcgagcgttctc

SEQ ID NO: 2 - DNA, 60 bp, artificial sequence
  synthetic sequence K3-dA40
  phosphorothioate linkage (1)..(59)
atcgactctcgagcgttctcaaaaaaaaaaaaaaaaaaaaaaaaaaaaaaaaaaaaaaaa

SEQ ID NO: 3 - DNA, 60 bp, artificial sequence
  synthetic sequence dA40-K3
  phosphorothioate linkage (1)..(59)
aaaaaaaaaaaaaaaaaaaaaaaaaaaaaaaaaaaaaaaaatcgactctcgagcgttctc

SEQ ID NO: 4 - DNA, 40 bp, artificial sequence
  synthetic sequence K3-dA20
  phosphorothioate linkage (1)..(39)
atcgactctcgagcgttctcaaaaaaaaaaaaaaaaaaaa

SEQ ID NO: 5 - DNA, 45 bp, artificial sequence
  synthetic sequence K3-dA25
  phosphorothioate linkage (1)..(44)
atcgactctcgagcgttctcaaaaaaaaaaaaaaaaaaaaaaaaa

SEQ ID NO: 6 - DNA, 50 bp, artificial sequence
  synthetic sequence K3-dA30
  phosphorothioate linkage (1)..(49)
atcgactctcgagcgttctcaaaaaaaaaaaaaaaaaaaaaaaaaaaaaa

SEQ ID NO: 7 - DNA, 55 bp, artificial sequence
  synthetic sequence K3-dA35
  phosphorothioate linkage (1)..(54)
atcgactctcgagcgttctcaaaaaaaaaaaaaaaaaaaaaaaaaaaaaaaaaaa

(19) United States
(12) Patent Application Publication
(10) Pub. No.: US 2018/0256630 A1
(43) Pub. Date: Sep. 13, 2018

(54) USE OF NUCLEIC ACID-POLYSACCHARIDE COMPLEXES HAVING IMMUNOPOTENTIATING ACTIVITY AS ANTI-TUMOR DRUG

(71) Applicant: National Institutes of Biomedical Innovation, Health and Nutrition, Ibaraki-shi, Osaka (JP)

(72) Inventors: Ken Ishii, Ibaraki-shi, Osaka (JP); Taiki Aoshi, Ibaraki-shi, Osaka (JP); Kouji Kobiyama, Ibaraki-shi, Osaka (JP)

(21) Appl. No.: 15/539,627

(22) PCT Filed: Dec. 26, 2014
(86) PCT No.: PCT/JP2014/084772

§ 371 Date: Jun. 23, 2017

(51) Int. Cl. A61K 31/7125; A61K 31/716; A61P 35/00

(52) U.S. Cl. CPC A61K 31/7125; A61P 35/00; A61K 31/716

(57) ABSTRACT

The present invention provides an anticancer agent to be used as a single agent. More specifically, the present invention provides an anticancer agent containing complexes that contain (a) an oligodeoxynucleotide containing a humanized K-type CpG oligodeoxynucleotide and polydeoxyadenylic acid, the polydeoxyadenylic acid being located on the 3′ side of the humanized K-type CpG oligodeoxynucleotide, and (b) β-1,3-glucan. The present invention is preferably characterized in that the anticancer agent is administered without a cancer antigen.

### TECHNICAL FIELD

The present invention relates to novel cancer therapy.

CpG oligonucleotides (CpG ODN) are short (about 20 base pairs) single-stranded synthetic DNA fragments comprising an immunostimulatory CpG motif. A CpG oligonucleotide is a potent agonist of Toll-like receptor 9 (TLR9), which activates dendritic cells (DCs) and B cells to produce type I interferons (IFNs) and inflammatory cytokines (Non Patent Literatures 1 and 2), and acts as an adjuvant of Th1 humoral and cellular immune responses including cytotoxic T lymphocyte (CTL) responses (Non Patent Literatures 3 and 4). In this regard, CpG ODNs has been considered to be a potential immunotherapeutic agent against infections, cancer, asthma, and hay fever (Non Patent Literatures 2 and 5).

There are at least four types of CpG ODNs, each with a different backbone sequence and immunostimulatory properties (Non Patent Literature 6). D type (also called A type) CpG ODNs typically comprise a CpG motif of a palindromic structure with a phosphodiester (PO) backbone and a phosphorothioate (PS) poly-G tail, and activate and induce plasmacytoid dendritic cells (pDCs) to produce a large quantity of IFN-α, but cannot induce pDC maturation or B cell activation (Non Patent Literatures 7 and 8). The other three types of ODNs consist of a PS backbone. K type (also called B type) CpG ODNs typically contain multiple CpG motifs with a non-palindromic structure, and can potently activate B cells to induce IL-6 production and activate pDCs to induce their maturation, but induce hardly any IFN-α production (Non Patent Literatures 8 and 9). Recently developed C and P type CpG ODNs comprise one and two palindromic structure CpG sequences, respectively. Both can activate B cells, like K type CpG ODNs, and activate pDCs, like D type CpG ODNs. Meanwhile, C type CpG ODNs more weakly induce IFN-α production relative to P type CpG ODNs (Non Patent Literatures 10 to 12). Patent Literature 1 describes many excellent K type CpG ODNs.

D type and P type CpG ODNs are shown to form a high-order structure i.e., Hoogsteen base pair forming a four parallel strand structure called G-tetrads and Watson-Crick base pair between a cis palindromic structure site and a trans palindromic structure site, respectively, which are required for potent IFN-α production by pDCs (Non Patent Literature 12 to 14). Such high order structures appear to be required for localization to initial endosomes or information transmission via TLR9, but they are affected by polymorphism and precipitation of the product, resulting in obstruction of clinical applications (Non Patent Literature 15). Thus, only K type and C type CpG ODNs are generally usable as immunotherapeutic agents and vaccine adjuvants for humans (Non Patent Literatures 16 and 17). K type CpG ODNs increase immunogenicity of vaccines targeting infections and cancer in human clinical trials (Non Patent Literatures 6 and 16), but a chemical and physical link between an antigen and K type CpG ODN is required for the optimal adjuvant effect. These results indicate that the four types (K, D, P, and C) of CpG ODNs have advantages and disadvantages. Thus, development of an “all-in-one” CpG ODN that can activate both B cells and pDCs without aggregation is desired.

Schizophyllan (SPG), which is a soluble β-1,3-glucan derived from Schizophyllum commune, is a medicament that has been approved for over 30 years in Japan as a stimulant for radiation therapy in cervical cancer patients (Non Patent Literature 18). Similarly, lentinan (LNT), which is a soluble β-1,3-glucan derived from shiitake mushrooms, is a medicament approved in 1985 that is used concomitantly with a fluoropyrimidine-based agent for patients with inoperable or recurrent gastric cancer (Non Patent Literatures 19 and 20). β-1,3-glucan is shown to form a complex having a triple helix structure with polydeoxyadenylic acid (dA) (Non Patent Literature 21).

Patent Literatures 2 to 4 disclose the use of a water-soluble complex of β-1,3-glucan including schizophyllan and a nucleic acid (gene) as a gene carrier. These documents describe that formation of such a complex enhances the resistance to a nuclease and antisense action of a gene.

Patent Literature 5 discloses that use of polysaccharides with a β-1,3-bond as a carrier (transfection agent) enhances the action of an immunostimulatory oligonucleotide comprising a CpG sequence and having a phosphodiester bond replaced with a phosphorothioate bond or a phosphorodithioate bond.

Patent Literature 6 describes an immunostimulatory complex characterized by consisting of an immunostimulatory oligonucleotide and a β-1,3-glucan having a long chain β-1,6-glucoside bond side chain.

The inventors have previously demonstrated that mouse and humanized CpG ODNs linked with poly(dA) having a phosphodiester bond at the 5′ end, which were complexed with SPG, enhance cytokine production and act as an influenza vaccine adjuvant or a prophylactic or therapeutic agent for Th2 cell related diseases (Non Patent Literatures 22 and 23 and Patent Literature 7). When poly (dA) was added to the 5′ end of each of K type and D type CpGs and a complex with SPG was formed, the properties of each of K type and D type were maintained while the activity thereof was enhanced. However, it has been difficult to achieve high yield of CpG-SPG complexes for preclinical and clinical development more efficiently and at a higher cost-effectiveness. It has been recently demonstrated that complex formation increases to almost 100% when poly (dA) with a phosphorothioate bond is linked to CpG ODNs (Non Patent Literature 24). However, an elaborate test has not been conducted for identifying the optimal humanized CpG sequence and optimizing an agent for obtaining “all-in-one” activity of four types of CpG ODNs.

Patent Literature 8 discloses a method of manufacturing an antigen/CpG oligonucleotide/β-1,3-glucan-based three-dimensional complex.

Synthetic nucleic acid CpG oligodeoxynucleotides (CpG ODNs), which are ligands of a Toll-like receptor 9 (TLR9), have potent innate immune activating capability and have expectations as a vaccine adjuvant. Since CpG ODNs have antitumor activity even in monotherapeutic administration, CpG ODNs also have expectations as an immunotherapeutic agent for cancer. However, while conventional CpG ODNs have antitumor activity, the effect can be exerted only by direct administration to tumor. Thus, clinical application thereof was considered difficult. In fact, it is considered difficult to directly administer an agent to tumor at an early stage in clinical settings. Further, clinical application is challenging at deeper sites, as surgical procedure would be required.

The inventors have recently developed a novel TLR9 ligand (K3-SPG), which is a CpG ODN wrapped with a polysaccharide beta glucan (PCT application (PCT/JP2014/074835)). K3-SPG is shown to have a more potent innate immunity activation compared to conventional CpG ODNs without forming an aggregate mass, in addition to a potent adjuvant effect, by an experiment using mice. It has been also revealed that K3-SPG induces potent acquired immunity in not only mice but cynomolgus monkeys, overcoming the difference in reactivity in mice and primates, which had been a concern up to this point.

In this manner, this CpG ODN has expectations in applications as an adjuvant agent, but it is unclear whether the CpG ODN can be used alone as a medicament.

For cancer therapy, since cancer related antigens recognized by cytotoxic T cells have been identified and reported in 1991 (Non Patent Literature 25=van der Bruggen et al. Science (New York, N.Y.) 254, 1643-1647 (1991)), many cancer related antigens have been identified at the molecular level, and clinical applications of cancer immunotherapy targeting them have been achieved (Non Patent Literature 26=Jager, E., et al. The Journal of experimental medicine 187, 265-270 (1998); Non Patent Literature 27=Jager, D. et al. Journal of clinical pathology 54, 669-674 (2001); Non Patent Literature 28=Imai, K., et al. British journal of cancer 104, 300-307 (2011); Non Patent Literature 29=Kang, X., et al. The Journal of Immunology 155, 1343-1348 (1995)). Cancer immunotherapy of particular note is the cancer vaccine Provenge, which uses antigen presenting cells of self-peripheral blood and received approveal from the US Food and Drug Administration (FDA) for prostate cancer patients for the first time in April 2010 (Non Patent Literature 30=Cancer vaccine approval could open floodgates. Nature medicine 16, 615-615 (2010); Non Patent Literature 31=Higano, C. S., et al. Cancer 115, 3670-3679 (2009)). Subsequently, Ipilimumab, which is an inhibitory antibody against cytotoxic T-lymphocyte antigen 4 (CTLA-4) that is an inhibitory molecule for T lymphocyte activation, was approved for malignant melanoma patients in the US in May 2011 (Non Patent Literature 32=Phan, G. Q., et al. Proc. Natl. Acad. Sci. U.S.A. 100, 8372-8377 (2003); Non Patent Literature 33=Camacho, L. H., et al. Journal of clinical oncology: official journal of the American Society of Clinical Oncology 27, 1075-1081 (2009); Non Patent Literature 34=Hodi, F. S., et al. New England Journal of Medicine 363, 711-723 (2010)). Furthermore, nivolumab, which is an immunoreaction inhibitory factor PD-1 (programmed cell death 1) response inhibitor, is in a clinical trial stage (Non Patent Literature 35=AZIJLI, K., et al. Anticancer Research 34, 1493-1505 (2014); Non Patent Literature 36=Okazaki, T., et al. Nature immunology 14, 1212-1218 (2013); Non Patent Literature 37=Ishida, Y., et al. The EMBO journal 11, 3887-3895 (1992); Non Patent Literature 38=Topalian, S. L., et al. The New England journal of medicine 366, 2443-2454 (2012)).

The environmental formation for which a dendritic cell guides an anti-cancer effector effectively is required for the pattern molecule of innate immunity to demonstrate the anti-cancer effect at the place of inflammation (Non Patent Literature 39=Chiba, S., et al. Nature immunology 13, 832-842 (2012)). Although the group of molecules described above and the course associated therewith have not been identified, dendritic cells activate lymphocytes such as CD4, CD8, and NK (Non Patent Literature 40=Engelhardt, J. J., et al. Cancer cell 21, 402-417 (2012)). Tumor infiltrating macrophages (tumor associated macrophage: TAM) are known as the cause of inflammatory reactions (Non Patent Literature 41=Huang, Y., et al. Cancer cell 19, 1-2 (2011)). Meanwhile, dendritic cells that function as the playmaker of anticancer immunity are also myeloid cells, just like microphages (Non Patent Literature 42=Huang, Y., et al. Proc. Natl. Acad. Sci. U.S.A. 109, 17561-17566 (2012)). A method that changes them to anticancer directivity has yet to be found, but tumor is understood to have avoided immunity by complex factors. Both TAM and dendritic cells are governed by inflammation and pattern recognition responses (Non Patent Literature 43=Garaude, J., et al. Science translational medicine 4, 120ra116 (2012); Non Patent Literature 44=Martinez-Pomares, L. et al. Trends in immunology 33, 66-70 (2012)). It is essential that immunological effector cells contact cancer cells in order to attack the cancer cells (Non Patent Literature 40=Engelhardt, J. J., et al. Cancer cell 21, 402-417 (2012); Non Patent Literature 45=Palucka, K. et al. Nature reviews. Cancer 12, 265-277 (2012)).

### CITATION LIST

### Patent Literature

[PTL 1] U.S. Pat. No. 8,030,285 B2
[PTL 2] WO 01/034207 A1
[PTL 3] WO 02/072152 A1
[PTL 4] Japanese Laid-Open Publication No. 2004-107272
[PTL 5] WO 2004/100965 A1
[PTL 6] Japanese Laid-Open Publication No. 2007-70307
[PTL 7] Japanese Laid-Open Publication No. 2008-100919
[PTL 8] Japanese Laid-Open Publication No. 2010-174107

### Non Patent Literature

[NPL 1] Hemmi, H., et al. Nature 408, 740-745 (2000).
[NPL 2] Krieg, A. M. Nature reviews. Drug discovery 5, 471-484 (2006).
[NPL 3] Brazolot Millan, C. L., et al., Proceedings of the National Academy of Sciences of the United States of America 95, 15553-15558 (1998).
[NPL 4] Chu, R. S., et al., The Journal of experimental medicine 186, 1623-1631 (1997).
[NPL 5] Klinman, D. M. Nature reviews. Immunology 4, 249-258 (2004).
[NPL 6] Vollmer, J. & Krieg, A. M. Advanced drug delivery reviews 61, 195-204 (2009).
[NPL 7] Krug, A., et al. European journal of immunology 31, 2154-2163 (2001).
[NPL 8] Verthelyi, D., et al., Journal of immunology 166, 2372-2377 (2001).
[NPL 9] Hartmann, G. & Krieg, A. M. Journal of immunology 164, 944-953 (2000).
[NPL 10] Hartmann, G., et al. European journal of immunology 33, 1633-1641 (2003).
[NPL 11] Marshall, J. D., et al. Journal of leukocyte biology 73, 781-792 (2003).
[NPL 12] Samulowitz, U., et al. Oligonucleotides 20, 93-101 (2010).
[NPL 13] Kerkmann, M., et al. The Journal of Biological Chemistry 280, 8086-8093 (2005).
[NPL 14] Klein, D. C., et al., Ultramicroscopy 110, 689-693 (2010).
[NPL 15] Puig, M., et al. Nucleic acids research 34, 6488-6495 (2006).
[NPL 16] Bode, C., et al., Expert review of vaccines 10, 499-511 (2011).
[NPL 17] McHutchison, J. G., et al. Hepatology 46, 1341-1349 (2007).
[NPL 18] Okamura, K., et al. Cancer 58, 865-872 (1986).
[NPL 19] Oba, K. et al., J. Individual patient based meta-analysis of lentinan for unresectable/recurrent gastric cancer. Anticancer Res., 2009, 29, 2739-2746.
[NPL 20] Nakano, H. et al., Hepato-Gastroenterol., 1999, 46, 2662-2668.
[NPL 21] Sakurai, K., et al., Biomacromolecules 2, 641-650 (2001).
[NPL 22] Shimada, N., et al. Bioconjugate chemistry 18, 1280-1286 (2007).
[NPL 23] Koyama, S., et al. Science translational medicine 2, 25ra24 (2010).
[NPL 24] Minari, J., et al. Bioconjugate chemistry 22, 9-15 (2011).
[NPL 25] van der Bruggen et al. Science (New York, N.Y.) 254, 1643-1647 (1991)
[NPL 26] Jager, E., et al. The Journal of experimental medicine 187, 265-270 (1998)
[NPL 27] Jager, D. et al. Journal of clinical pathology 54, 669-674 (2001).
[NPL 28] Imai, K., et al. British journal of cancer 104, 300-307 (2011)
[NPL 29] Kang, X., et al. The Journal of Immunology 155, 1343-1348 (1995)
[NPL 30] Cancer vaccine approval could open floodgates. Nature medicine 16, 615-615 (2010)
[NPL 31] Higano, C. S., et al. Cancer 115, 3670-3679 (2009)
[NPL 32] Phan, G. Q., et al. Proc. Natl. Acad. Sci. U.S.A. 100, 8372-8377 (2003)
[NPL 33] Camacho, L. H., et al. Journal of clinical oncology: official journal of the American Society of Clinical Oncology 27, 1075-1081 (2009)
[NPL 34] Hodi, F. S., et al. New England Journal of Medicine 363, 711-723 (2010)
[NPL 35] AZIJLI, K., et al. Anticancer Research 34, 1493-1505 (2014)
[NPL 36] Okazaki, T., et al. Nature immunology 14, 1212-1218 (2013)
[NPL 37] Ishida, Y., et al. The EMBO journal 11, 3887-3895 (1992)
[NPL 38] Topalian, S. L., et al. The New England journal of medicine 366, 2443-2454 (2012)
[NPL 39] Chiba, S., et al. Nature immunology 13, 832-842 (2012)
[NPL 40] Engelhardt, J. J., et al. Cancer cell 21, 402-417 (2012)
[NPL 41] Huang, Y., et al. Cancer cell 19, 1-2 (2011)
[NPL 42] Huang, Y., et al. Proc. Natl. Acad. Sci. U.S.A. 109, 17561-17566 (2012)
[NPL 43] Garaude, J., et al. Science translational medicine 4, 120ra116 (2012)
[NPL 44] Martinez-Pomares, L. et al. Trends in immunology 33, 66-70 (2012)
[NPL 45] Palucka, K. et al. Nature reviews. Cancer 12, 265-277 (2012)

### SUMMARY OF INVENTION

### Solution to Problem

As a result of detailed studies, the inventors have completed the present invention by using a CpG-β glucan complex (e.g., K3-SPG (complex of human K type CpG ODN, K3, and beta glucan)), which had been developed as a conventional adjuvant, as an antitumor drug as a monotherapeutic to confirm tumor regression in tumor of cancer-bearing mice with K3-SPG in intravenous administration, which was ineffective with conventional CpG ODNs (K3) (FIG. 2 (A-B)). The inventors further demonstrated that a potent antitumor activity is exhibited in peritoneal seeding model, which is a model closer to clinical settings (FIGS. 2g and m (FIG. 2B)). The inventors have confirmed that this effect does not require administration of antigens, and an effect is exhibited in administration as a monotherapeutic.

Furthermore, the inventors have shown, using gene knockout mice, that acquired immune responses are important for the antitumor effect of K3-SPG, and IL-12 and type I interferon (IFN) induced by innate immune responses are essential (FIGS. 6a, b, and c (FIG. 6A)). The inventors have also confirmed that CD45 negative tumor cells accumulate in the spleen by intravenous administration of K3-SPG and revealed that many such cells undergo necrosis or apoptosis. When mice were immunized with these CD45 negative cells, potent antitumor effect was exhibited, revealing that cell death of CD45 negative cells accumulated in the spleen may be playing an important role (FIGS. 6g, h, i, and j (FIG. 6B)). The inventors have also confirmed that activated CD8 T cells accumulate in tumor by administering K3-SPG, revealing that these cells are essential for an antitumor effect.

For this reason, a potent effect is expected even in carcinoma for which development of CpG ODNs that exerts an antitumor effect in systemic administration had been difficult. Furthermore, CpG ODNs exert an antitumor effect without antigens, such that there is expectation for their application as a monotherapy.

Up to this point, CpG ODNs are shown to be a promising drug for monotherapy (Pratesi, G., et al. Cancer research 65, 6388-6393 (2005); Manegold, C., et al. Annals of oncology: official journal of the European Society for Medical Oncology/ESMO 23, 72-77 (2012); Kim, Y. H., et al. Blood 119, 355-363 (2012); Hirsh, V., et al. Journal of clinical oncology: official journal of the American Society of Clinical Oncology 29, 2667-2674 (2011); Weber, J. S., et al. Cancer 115, 3944-3954 (2009)) or as a cancer vaccine adjuvant (Reed, S. G., Nature medicine 19, 1597-1608 (2013); Perret, R., et al. Cancer research 73, 6597-6608 (2013); Mbow, M. L., et al. Current opinion in immunology 22, 411-416 (2010); Duthie, M. S., et al. Immunological reviews 239, 178-196 (2011)). However, conventional therapy with CpG-ODNs as an anticancer agent can suppress tumor growth only when injected into tumor (Schettini, J., et al. Cancer immunology, immunotherapy: CII 61, 2055-2065 (2012); Lin, A. Y., et al. PLoS One 8, e63550 (2013); Ishii, K. J., et al. Clinical cancer research: an official journal of the American Association for Cancer Research 9, 6516-6522 (2003); Lou, Y., et al. Journal of immunotherapy (Hagerstown, Md.: 1997) 34, 279-288 (2011); Auf, G., Clinical cancer research: an official journal of the American Association for Cancer Research 7, 3540-3543 (2001); Nierkens, S., et al. PLoS One 4, e8368 (2009); Heckelsmiller, K., et al. Journal of immunology 169, 3892-3899 (2002)). In this regard, the inventors have developed nanoparticle-like TLR9 agonist, K3-SPG, which consists of a schizophyllan (SPG; β glucan) and B/K type CpG (K3) complex, and demonstrated that K3-SPG functioned as a stronger vaccine adjuvant (involving potent induction of IFN-α) than the original K3. In the Examples, the inventors further investigated the potential of K3-SPG in monotherapeutic immunotherapy on cancer (that does not use additional tumor peptides or antigens) to find that the above-described effect is obtained to complete the present invention. Thus, the present invention typically provides the following.

(Monotherapeutic Anticancer Agent)

(1) An anticancer agent comprising a complex, comprising:
(a) an oligodeoxynucleotide comprising a humanized K type CpG oligodeoxynucleotide and polydeoxyadenylic acid, wherein the polydeoxyadenylic acid is disposed on the 3′ side of the humanized K type CpG oligodeoxynucleotide; and
(b) β-1,3-glucan.
(2) The anticancer agent of item (1), characterized in that the anticancer agent is administered without a cancer antigen.
(3) The anticancer agent of item (1) or (2), characterized in that the anticancer agent is administered to be delivered to a reticuloendothelial system and/or a lymph node.
(4) The anticancer agent of item (3), wherein the reticuloendothelial system and/or lymph node comprises tumor and phagocytes.
(5) The anticancer agent of item (3) or (4), wherein the reticuloendothelial system comprises a spleen and/or a liver.
(6) The anticancer agent of any one of items (1) to (5), wherein the anticancer agent is administered without a cancer antigen.
(7) The anticancer agent of any one of items (2) to (6), wherein the administration comprises systemic administration.
(8) The anticancer agent of item (7), wherein the systemic administration is selected from intravenous administration, intraperitoneal administration, oral administration, subcutaneous administration, intramuscular administration, or intratumoral administration.
(9) The anticancer agent of any one of items 1 to 8, wherein the oligodeoxynucleotide is selected from the group consisting of K3 (SEQ ID NO: 1), K3-dA₄₀ (SEQ ID NO: 2), dA₄₀-K3 (SEQ ID NO: 3), K3-dA20 (SEQ ID NO: 4), K3-dA25 (SEQ ID NO: 5), K3-dA30 (SEQ ID NO: 6), and K3-dA35 (SEQ ID NO: 7).
(10) The anticancer agent of any one of items 1 to 9, wherein the β-1,3-glucan is selected from the group consisting of schizophyllan (SPG), lentinan, scleroglucan, curdlan, pachyman, grifolan, and laminaran.
(11) The anticancer agent of any one of items 1 to 10, wherein the complex is K3-SPG.
(Agent Inducing Accumulation in Reticuloendothelial System (Including the Spleen and/or Liver) and/or Lymph Node)
(12) A composition for inducing accumulation of dead cancer cells in a spleen, comprising a complex comprising:
(a) an oligodeoxynucleotide comprising a humanized K type CpG oligodeoxynucleotide and polydeoxyadenylic acid, wherein the polydeoxyadenylic acid is disposed on the 3′ side of the humanized K type CpG oligodeoxynucleotide; and
(b) β-1,3-glucan.
(13) The composition of item (12), wherein the oligodeoxynucleotide is selected from the group consisting of K3 (SEQ ID NO: 1), K3-dA₄₀ (SEQ ID NO: 2), dA₄₀-K3 (SEQ ID NO: 3), K3-dA20 (SEQ ID NO: 4), K3-dA25 (SEQ ID NO: 5), K3-dA30 (SEQ ID NO: 6), and K3-dA35 (SEQ ID NO: 7).
(14) The composition of item (12) or (13), wherein the β-1,3-glucan is selected from the group consisting of schizophyllan (SPG), lentinan, scleroglucan, curdlan, pachyman, grifolan, and laminaran.
(15) The composition of any one of items (12) to (14), wherein the complex is K3-SPG.
(16) The composition of any one of items 12 to 15, wherein the reticuloendothelial system and/or lymph node comprises tumor and phagocytes.
(17) The composition of any one of items (12) to (16), wherein the reticuloendothelial system comprises a spleen and/or a liver.
(18) The composition of any one of items (12) to (17), wherein the administration comprises systemic administration.
(19) The composition of item (18), wherein the systemic administration is selected from intravenous administration, intraperitoneal administration, oral administration, subcutaneous administration, intramuscular administration, or intratumoral administration.
<Composition for the Expression of Interleukin 12 (IL12) and/or Interferon (IFN) γ or the Enhancement Thereof>
(20) A composition for the expression of interleukin 12 (IL12) and/or interferon (IFN) γ or the enhancement thereof, comprising:
(a) an oligodeoxynucleotide comprising a humanized K type CpG oligodeoxynucleotide and polydeoxyadenylic acid, wherein the polydeoxyadenylic acid is disposed on the 3′ side of the humanized K type CpG oligodeoxynucleotide; and
(b) β-1,3-glucan.
(21) The composition of item (20), wherein the oligodeoxynucleotide is K3 (SEQ ID NO: 1), K3-dA₄₀ (SEQ ID NO: 2), dA₄₀-K3 (SEQ ID NO: 3), K3-dA20 (SEQ ID NO: 4), K3-dA25 (SEQ ID NO: 5), K3-dA30 (SEQ ID NO: 6), and K3-dA35 (SEQ ID NO: 7).
(22) The composition of item (20) or (21), wherein the β-1,3-glucan is selected from the group consisting of schizophyllan (SPG), lentinan, scleroglucan, curdlan, pachyman, grifolan, and laminaran.
(23) The composition of any one of items (20) to (22), wherein the complex is K3-SPG.

In the present invention, one or more of the features described above are intended to be provided not only as the explicitly described combinations, but also as other combinations thereof. The additional embodiments and advantages of the present invention are recognized by those skilled in the art by reading and understanding the following detailed description, as needed.

### Advantageous Effects of Invention

The application of the present invention K3-SPG as an antitumor drug can exert a potent antitumor effect in systemic administration, which was not possible with conventional CpG ODNs. For this reason, the present invention is also considered very useful from the clinical viewpoint. Since K3-SPG is also confirmed to have a sufficient effect (innate immune response) in human cells, the possibility for human application is high. Since the inventors' research results demonstrate that K3-SPG has a more potent antitumor effect, in addition to a much higher innate immune activating capability, compared to conventional CpG ODNs that have been used in clinical trials, K3-SPG has expectations as a useful immunotherapeutic drug. Furthermore, K3-SPG can exert an effect by inducing tumor cell death without administration of an antigen, such that K3-SPG is considered to be applicable to various carcinomas. In view of these results, K3-SPG has potential as an innate immune activating antitumor drug that does not require an antigen.

### BRIEF DESCRIPTION OF DRAWINGS

FIG. 1 shows a method of conjugating a CpG ODN with SPG.

FIG. 2 (A-B) shows that systemic injection of antigen-free nanoparticle-like CpGs (K3-SPG) can be applied to many established tumor models including pancreatic cancer peritoneal seeding models. FIG. 2A shows a to i. C57BL/6 mice were s.c. inoculated with EG7 cells on day 0, and PBS (a-c), K3 (30 μg) (d-f), or K3-SPG (10 μg) (g-i) was intradermally (i.d.) (surrounding region of tumor) (a, d, and g), intratumorally (i.t.) (b, e, and h), or intravenously (i.v.) (c, f, and 1) administered on days 7, 9, and 11. The tumor size was measured for 23 days (n=4). Each curve represents an individual mouse. The arrows indicate the timing of therapy.

FIG. 2 (A-B) shows that systemic injection of antigen-free nanoparticle-like CpGs (K3-SPG) can be applied to many established tumor models including pancreatic cancer peritoneal seeding models. FIG. 2B shows j to n. (j to l) C57BL/6 mice were inoculated with B16 cells, B16F10 cells, or MC38 cells on day 0. The B16 inoculation group was i.v. or i.t. treated with K3-SPG on day 10, 12, and 14. The B16F10 inoculation group was i.v. or i.t. treated with K3-SPG on days 7, 9, and 11. The MC38 inoculation group was i.v. or i.t. treated with K3-SPG on day 14, 16, and 18. The error bar represents mean+SEM (n=4). *p<0.05 (t-test). (m) C57BL/6 mice were intraperitoneally injected with Pan02 cells on day 0 and i.v. treated with K3, K3-SPG, or PBS (control) on day 11, 13, and 15. The tumor weight (g) shown is for day 21. *p<0.05 (t-test). (n) C57BL/6 mice were intraperitoneally injected with Pan02 on day 0 and i.v. treated i.v. three times with K3, K3-SPG, or PBS. The survival (%) is shown (n=8). *p<0.05 (log-rank test).

FIG. 3 is a comparison of results from systemic administration of K3-SPG with that for another control and K3. It is demonstrated that K3-SPG that is systemically administered can be a cancer immunotherapeutic agent, which does not require antigens. The tumor cell line EG7 was transplanted into mice, and then K3 or K3-SPG was intravenously administered three times (days 7, 9, and 11). The tumor size was measured 7 days from the transplantation of tumor cells.

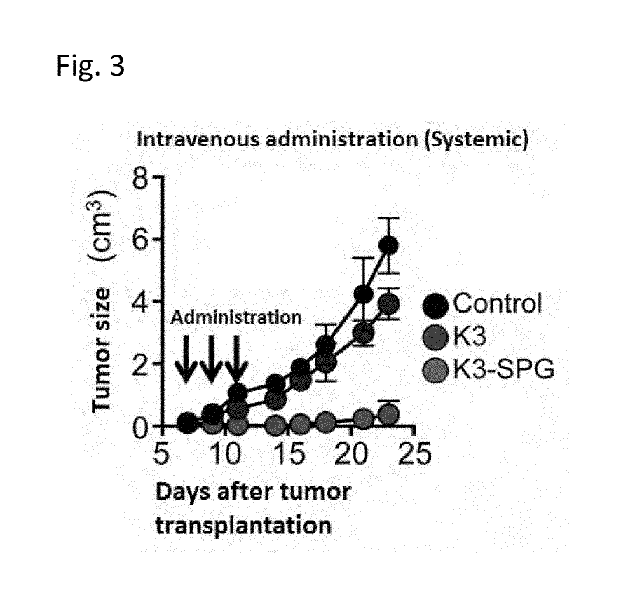

FIG. 4 shows that K3-SPG targets phagocytes in a tumor microenvironment. (a to c) C57BL/6 mice were s.c. inoculated with EG7 on day 0. PBS (control), ALEXA 647-K3 (30 μg), or ALEXA 647-K3-SPG (10 μg) was i.v. administered on day 12. 1 hour after administration, the mice were analyzed with an in vivo fluorescence imaging system (IVIS). Images measured by relative fluorescence were converted into a unit or measurement of surface radiance (photons/sec/cm²/sr). The white arrows indicate tumor inoculation region (a). (b and c) Frozen sections of tumor from FIG. 6a (6A) were stained with anti-CD3e antibodies (red, EG7 staining) and Hoechst 33258 (blue, nuclear staining), and were analyzed thereafter with a fluorescence microscope (scale bar, 100 μm). The white arrows indicate fluorescence positive regions. (d to g) C57BL/6 mice were s.c. inoculated with EG7 on day 0. Alexa 647-K3, Alexa 647-K3-SPG, or FITC-SPG was i.v. administered on day 12 with dextran-PE. (d to f) One hour after the injection, the frozen sections of tumor were analyzed with a fluorescence microscope (scale bar, 100 μm). (g) Green, red, or merged cells were counted (10 fields each from 3 tumors). The error bar represents mean+SD. The asterisks indicate a significant difference from K3-injected merged cell count. (h) C57BL/6 mice (n=3 or 4) were s.c. inoculated with EG7 on day 0. On day 5, a clodronate liposome or a control liposome was i.v. administered. Mice were injected with PBS (control) or K3-SPG on days 7, 9, and 11. The error bar represents mean+SEM. The arrows indicate the timing of therapy *p<0.05 (t-test).

FIG. 5 shows that F4/80 positive cells in tumor were depleted by clodronate liposomes. C57BL/6 mice were inoculated with EG7 on day 0. Clodronate liposomes (a) or control liposomes (b) were i.v. administered on day 5 and i.v. treated with Alexa 647-K3-SPG on day 7. One hour after the treatment, frozen sections of tumor were stained with anti-F4/80 antibodies (red) and Hoechst 33258 (blue) and then were analyzed with a fluorescence microscope (scale bar, 100 μm).

FIG. 6 (A-B) shows that both IL-12 and IFN have a potentially important role in tumor regression and immunogenic cell death thereof. FIG. 6A shows a to f. (a to c) Il12p40 hetero knockout mice (a), Ifnar2 hetero knockout mice (b), and Il12p40-Ifnar2 double knockout mice (c) were s.c. inoculated with EG7 cells on day 0, and the mice were i.v. treated with K3-SPG on days 7, 9, and 11. The error bar represents mean+SEM (n=4). The arrows show the timing of therapy. *p<0.05 (t-test). (d and f) Rag2 hetero and knockout mice, and Il12p40-Ifnar2 double knockout mice were inoculated with EG7 cells on day 0 and i.v. treated with K3-SPG three times (days 7, 9, and 11, black arrows), 6 times (days 7, 9, 11, 14, 16, and 18, gray arrows), or 0 times (control). (e) The expanded diagram shows day 4 to day 21.

FIG. 6 (A-B) shows that both IL-12 and IFN have a potentially important role in tumor regression and immunogenic cell death thereof. FIG. 6B shows g to k. (g) C57BL/6 mice and Il12p40-Ifnar2 double knockout mice were s.c. inoculated with EG7 cells on day 0, and the mice were i.v. treated with K3-SPG on days 7, 9, and 11. The mice were then slaughtered on day 12. Splenocytes were collected and stained with anti-CD45 antibodies, and then the cells were analyzed by flow cytometry. (h) The scatter diagram shows CD45-negative cell populations. The error bar represents mean+SEM. *p<0.05 (t-test). (1) CD45 negative populations were stained with PI and Hoechst 33342 for staining dead cells, and were analyzed thereafter by flow cytometry. The bar graphs indicate populations of apoptotic cells, necrotic cells, and CD45-negative live cells. The error bar represents mean+SD (n=3). *p<0.05 (t-test). (J) C57BL/6 mice were immunized with PBS or CD45 negative cells. Seven days after the immunization, mice were s.c. inoculated with EG7 cells on day 0. The tumor size was measured for the next 25 days (n=3). The error bar represents mean+SEM. *p<0.05 (t-test). (k) The tumor volume and the OVA₂₅₇₋₂₆₄ specific tetramer+CD8 T cell count on day 25 were each represented by a bar graph. *p<0.05 (t-test).

FIG. 7 shows that IFN-β was detected in a tumor microenvironment. (a) IFN-β GFP mice were inoculated with EG7 on day 0, and the mice were i.d. or i.v. treated with K3-SPG on days 7, 9, and 11. 12 days after the inoculation, tumor was collected. Frozen sections were stained with anti-CD11b antibodies, anti-CD169 antibodies, anti-F4/80 antibodies, anti-MARCO antibodies (red) and Hoechst 33258 (blue) and then analyzed with a fluorescence microscope (scale bar, 100 μm). (b) IFN-p positive cells were counted (10 fields each from 3 tumors). The error bar represents mean+SD. *p<0.05 (t-test).

FIG. 8 shows that IL12-p40 was detected in a tumor microenvironment. (a) C57BL/6 mice were inoculated with EG7 on day 0, and were i.d. or i.v. treated with K3-SPG on days 7, 9, and 11. 12 days after the inoculation, tumor was collected. Frozen sections were stained with anti-IL12-p40 antibodies (red) and Hoechst 33258 (blue) and then analyzed with a fluorescence microscope (scale bar, 100 μm). (b) IL12-p40 positive cells were counted (10 fields each from). The error bar represents mean+SD. *p<0.05 (t-test).

FIG. 9 shows that CD45 negative cells are derived from tumor cells, but not from host cells. GFP mice were s.c. inoculated with EG7 cells on day 0, and the mice were i.v. treated with K3-SPG on days 7, 9, and 11. The mice were then slaughtered on day 12. Splenocytes were collected and stained with anti-CD45 antibodies. The cells were then analyzed by flow cytometry.

FIG. 10 (A-B) shows that K3-SPG induced tumor regression requires both innate immune responses and adaptive immune responses, including I112, type 1 IFN, Batf3, CD8⁺ DC, and potent cytotoxic T cells that infiltrate tumor. FIG. 10A shows a to a. C57BL/6 knockout mice (a) and Batf3 hetero and Batf3 knockout mice (b) were inoculated with EG7 cells on day 0, and the mice were i.v. treated with K3-SPG on days 7, 9, and 11 (black arrows). (a) CD8 depleting antibodies (200 μg/mouse) were administered on day 6 and 13. The error bar represents mean+SEM (n=4). *p<0.05 (t-test). The arrows indicate the timing of therapy. (c) C57BL/6 mice were inoculated with EG7 on day 0, and the mice were i.d. or i.v. treated with K3-SPG on days 7, 9, and 11. 12 days after the inoculation, tumor was collected. Frozen sections were stained with anti-CD8β antibodies (red) and Hoechst 33258 (blue) and then analyzed with a fluorescence microscope (scale bar, 100 μm). CD8β positive cells were counted (10 fields each from). The error bar represents mean+SD. *p<0.05 (t-test).

FIG. 10 (A-B) shows that K3-SPG induced tumor regression requires both innate immune responses and adaptive immune responses, including I112, type 1 IFN, Batf3, CD8⁺ DC, and potent cytotoxic T cells that infiltrate tumor. FIG. 10B shows d to e. (d) C57BL/6 (WT) mice and Il12p40-Ifnar2 double-knockout (DKO) mice were inoculated with EG7 cells on day 0, and the mice were i.v. treated with K3-SPG on days 7, 9, and 11. CD8α⁺ T cells derived from tumor carrying mice injected with either K3-SPG or PBS were stained with Xenolight and transferred (i.v.) on day 14. On day 15, the mice were then analyzed by IVIS. (I and II) Recipient mice: EG7 carrying WT mice that were i.v. treated with K3-SPG. K3-SPG treated CD8α⁺ T cells (I) or untreated CD8α⁺ T cells (II) were transferred into the mice. (e) (I and II) Recipient mice: untreated EG7 carrying WT mice (I) and DKO mice i.v. treated with K3-SPG. K3-SPG treated CD8α⁺ T cells were transferred into the mice (I and II).

FIG. 11 shows a schematic diagram of the experimental system. WT mice and Il12p40-Ifnar2 DKO mice were inoculated with EG7 cells on day 0, and the mice were i.v. treated with K3-SPG or PBS on days 7, 9, and 11. On day 14, CD8α⁺ T cells were purified from the spleens of these mice and were labeled with Xenolight DiR®. The cells were transferred into another K3-SPG treated (days 7, 9, and 11) EG7 carrying mice (14 days after inoculation). The distribution of the Xenolight DiR®-labeled CD8 T cells was analyzed thereafter by IVIS on day 15.

FIG. 12 shows the strategy of K3-SPG treatment. K3-SPG targeted the tumor microenvironment through blood flow. In addition, K3-SPG targeted phagocytes, and activated these cells. In the tumor microenvironment, IFNs and IL-12 were induced by K3-SPG treatment. In addition, antigens were released through lymphatic flow and blood flow. Presentation of the antigens induced potent tumor-specific CTL.

### DESCRIPTION OF EMBODIMENTS

The present invention is explained hereinafter while disclosing the best mode of the invention. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form unless specifically noted otherwise. Further, the terms used herein should be understood as used in the meaning that is commonly used in the art, unless specifically noted otherwise. Thus, unless defined otherwise, all terminologies and scientific technical terms that are used herein have the same meaning as the general understanding of those skilled in the art to which the present invention pertains. In case of a contradiction, the present specification (including the definitions) takes precedence.

The definition of the terms and/or general techniques particularly used herein is explained hereinafter as appropriate.

The present invention provides an oligodeoxynucleotide comprising a K type CpG oligodeoxynucleotide and polydeoxyadenylic acid (dA) (hereinafter, referred to as the oligodeoxynucleotide of the invention). The oligodeoxynucleotide of the invention encompasses those with a phosphodiester bond that is modified (e.g., some or all of the phosphodiester bonds are substituted with a phosphorothioate bond). The oligonucleotide of the invention includes pharmaceutically acceptable salts.

As used herein, “CpG oligonucleotide (residue)” is interchangeably used with “CpG oligodeoxynucleotide (residue)”, “CpG ODN (residue)”, and simply “CpG (residue)” and refers to a polynucleotide, preferably an oligonucleotide, comprising at least one non-methylated CG dinucleotide sequence. The terms are synonymous regardless of the presence/absence of the term “residue” at the end. An oligonucleotide comprising at least one CpG motif can comprise multiple CpG motifs. As used herein, the phrase “CpG motif” refers to a non-methylated dinucleotide moiety of an oligonucleotide, comprising a cytosine nucleotide and the subsequent guanosine nucleotide. 5-methylcytosine may also be used instead of cytosine. Furthermore, polydeoxyadenylic acid is synonymous with polydeoxyadenosinic acid (residue). While the term “residue” refers to a partial structure of a compound with a larger molecular weight, as used herein, those skilled in the art can readily understand from the context as to whether “CpG oligodeoxynucleotide (CpG ODN)” refers to an independent molecule or a partial structure of a compound with a larger molecular weight. The same applies to terms related to other partial structures comprised by the oligodeoxynucleotide of the invention such as “polydeoxyadenylic acid”.

CpG oligonucleotides (CpG ODN) are short (about 20 base pairs) single-stranded synthetic DNA fragments comprising an immunostimulatory CpG motif. A CpG oligonucleotide is a potent agonist of Toll-like receptor 9 (TLR9), which activates dendritic cells (DCs) and B cells to induce type I interferons (IFNs) and inflammatory cytokine production (Hemmi, H., et al. Nature 408, 740-745 (2000); Krieg, A. M. Nature reviews. Drug discovery 5, 471-484 (2006).), and acts as an adjuvant of Th1 humoral and cellular immune responses including cytotoxic T lymphocyte (CTL) responses (Brazolot Millan, C. L., Weeratna, R., Krieg, A. M., Siegrist, C. A. & Davis, H. L. Proceedings of the National Academy of Sciences of the United States of America 95, 15553-15558 (1998); Chu, R. S., Targoni, O. S., Krieg, A. M., Lehmann, P. V. & Harding, C. V. The Journal of experimental medicine 186, 1623-1631 (1997)). In this regard, CpG ODNs were considered to be a potential immunotherapeutic agent against infections, cancer, asthma, and hay fever (Krieg, A. M. Nature reviews. Drug discovery 5, 471-484 (2006); Klinman, D. M. Nature reviews. Immunology 4, 249-258 (2004)).

A CpG oligodeoxynucleotide (CpG ODN) is a single stranded DNA comprising an immunostimulatory non-methylated CpG motif, and is an agonist of TLR9. There are four types of CpG ODNs, i.e., K type (also called B type), D type (also called A type), C type, and P type, each with a different backbone sequence and immunostimulatory properties (Advanced drug delivery reviews 61, 195-204 (2009)). The oligodeoxynucleotide of the invention comprises K type CpG ODNs thereamong.

Typically, K type CpG ODNs have structural and functional properties characterized by containing multiple CpG motifs with a non-palindromic structure and by inducing IL-6 production by activating B cells, but hardly inducing IFN-α production of plasmacytoid dendritic cells (pDCs). A non-methylated CpG motif refers to a short nucleotide sequence comprising at least one cytosine (C)-guanine (G) sequence whose cytosine is not methylated at position 5. In the following explanation, CpG refers to non-methylated CpG, unless specifically noted otherwise. Thus, inclusion of a K type CpG ODN results in immunostimulatory activity unique to K type CpG ODNs (e.g., activity to activate B cells (preferably human B cells) to induce IL-6 production). Many humanized K type CpG ODNs are known in the art (Journal of immunology 166, 2372-2377 (2001); Journal of immunology 164, 944-953 (2000); U.S. Pat. No. 8,030,285 B2).

K type CpG ODNs contained in the oligodeoxynucleotide of the invention are preferably humanized. “Humanized” refers to having agonistic activity against human TLR9. Thus, the oligodeoxynucleotide of the invention comprising a humanized K type CpG ODN has immunostimulatory activity unique to K type CpG ODNs against humans (e.g., activity to activate human B cells to induce IL-6 production). K type CpG ODNs suitably used in the present invention have a length of 10 nucleotides long or greater and comprise the nucleotide sequence set forth in the following formula:

5′N₁N₂N₃T-CpG-WN₄N₅N₆3′

wherein the middle CpG motif is not methylated, W is A or T, and N1, N2, N3, N4, N5, and N6 may be any nucleotide.

In one embodiment, the K type CpG ODN of the invention has a length of 10 nucleotides long or greater and comprises the nucleotide sequence of the above-described formula. However, in the above-described formula, the CpG motif of 4 bases in the middle (TCpGW) only needs to be included in the 10 nucleotides. The motif does not necessarily need to be positioned between N3 and N4 in the above-described formula. Further, the N1, N2, N3, N4, N5, and N6 may be any nucleotide in the above-described formula. Combinations of at least one (preferably one) of N1 and N2, N2 and N3, N3 and N4, N4 and N5, and N5 and N6 may be a two base CpG motif. When the four-base CpG motif is not positioned between N3 and N4, any two contiguous bases in the middle 4 bases (4th to 7th bases) in the above-described formula may be a CpG motif and the other two bases may be any nucleotide.

K type CpG ODNs suitably used in the present invention contain a non-palindromic structure comprising one or more CpG motifs. K type CpG ODNs more suitably used in the present invention consist of a non-palindromic structure comprising one or more CpG motifs.

Humanized K type CpG ODNs are generally characterized by a four base CpG motif consisting of TCGA or TCGT. In many cases, a single humanized K type CpG ODN comprises 2 or 3 of the four base CpG motifs. Thus, in a preferred embodiment, a K type CpG ODN contained in the oligodeoxynucleotide of the invention comprises at least 1, more preferably 2 or more, and still more preferably 2 or 3 four-base CpG motifs consisting of TCGA or TCGT. When such a K type CpG ODN has 2 or 3 four-base CpG motifs, these four base CpG motifs may be the same or different. However, this is not particularly limited, as long as there is agonist activity against human TLR9.

K type CpG ODNs comprised in the oligodeoxynucleotide of the invention more preferably comprise the nucleotide sequence set forth in SEQ ID NO: 1.

The length of K type CpG ODNs is not particularly limited, as long as the oligodeoxynucleotide of the invention activates immunostimulatory activity (e.g., activity to activate B cells (preferably human B cells) to induce IL-6 production), but the length is preferably 100 nucleotides long or less (e.g., 10 to 75 nucleotides long). The length of K type CpG ODNs is more preferably 50 nucleotides long or less (e.g., 10 to 40 nucleotides long). The length of K type CpG ODNs is still more preferably 30 nucleotides long or less (e.g., 10 to 25 nucleotides long). The length of K type CpG ODNs is most preferably 12 to 25 nucleotides long.

The length of polydeoxyadenylic acid (dA) is not particularly limited, as long as the length is sufficient to form a triple helix structure with a β-1,3-glucan (preferably lentinan or schizophyllan) chain. From the viewpoint of forming a stable triple helix structure, the length is generally 20 nucleotides long or greater, preferably 40 nucleotides long or greater, and more preferably 60 nucleotides long or greater. Since longer poly-dA forms a more stable triple helix structure with μ-1,3-glucan, there is theoretically no upper limit to the length. However, a length that is too long can be a cause of variability in the lengths upon oligodeoxynucleotide synthesis. Thus, the length is generally 100 nucleotides long or less, and preferably 80 or less. Meanwhile, from the viewpoint of increasing the amount of the oligodeoxynucleotide of the invention that binds to a unit quantity of β-1,3-glucan, avoidance of variability in the lengths in oligodeoxynucleotide synthesis, and complexing efficiency in addition to the aforementioned formation of stable triple helix structures, the length of poly-dA is preferably 20-60 nucleotides long (specifically, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides long), more preferably 30 to 50 nucleotides long (30, 31, 32, 33, 34, 35, 36, 37, 38, 39, 40, 41, 42, 43, 44, 45, 46, 47, 48, 49, or 50 nucleotides), and most preferably 30 to 45 nucleotides long (30, 31, 32, 33, 34, 35, 36, 37, 38, 39, 40, 41, 42, 43, 44, or 45 nucleotides long). In particular, when the length is 30 nucleotides long or greater, excellent complexing efficiency is exhibited. The oligodeoxynucleotide of the invention comprises poly-dA to have activity of forming a triple helix structure with two schizophyllan chains. It should be noted that polydeoxyadenylic acid may be denoted as “poly(dA)”.

The oligodeoxynucleotide of the invention of a single molecule may comprise multiple K type CpG ODNs and/or poly-dA, but preferably comprises one each of a K type CpG ODN and poly dA, and most preferably consists of one each of a K type CpG ODN and poly dA.

Examples of exemplary CpG sequences include, but are not limited to K3 CpG (SEQ ID NO: 1=5′-atcgactctcgagcgttctc-3′) and the like.

The oligodeoxynucleotide of the invention is characterized by poly dA being disposed on the 3′ side of K type CpG ODNs. The complex of the invention (details thereof discussed below) possibly has enhanced anticancer action due to such disposition, but the structure is not limited thereto. It may be bound anywhere as an anticancer agent.

A K type CpG ODN and poly-dA may be linked directly by a covalent bond or via a spacer sequence. A spacer sequence refers to a nucleotide sequence comprising one or more nucleotides inserted between two adjacent constituent elements. The length of a spacer sequence is not particularly limited, as long as the complex of the invention has immunostimulatory activity (preferably activity to activate B cells to induce IL-6 production and activity to activate dendritic cells to induce IFN-α production), but the length is generally 1 to 10 nucleotides long, preferably 1 to 5 nucleotides long, and more preferably 1 to 3 nucleotides long. Most preferably, a K type CpG ODN and poly-dA are directly linked by a covalent bond.

The oligodeoxynucleotide of the invention may have an additional nucleotide sequence at the 5′ end and/or the 3′ end in addition to a K type CpG ODN, poly-dA, and any spacer sequence. The length of the additional nucleotide sequence is not particularly limited, as long as the complex of the invention has immunostimulatory activity (preferably activity to activate B cells to induce IL-6 production and activity to activate dendritic cells to induce IFN-α production), but the length is generally 1 to 10 nucleotides long, preferably 1 to 5 nucleotides long, and more preferably 1 to 3 nucleotides long.

In a preferred embodiment, the oligodeoxynucleotide of the invention does not comprise such an additional nucleotide sequence at the 5′ end and/or 3′ end. That is, the oligodeoxynucleotide of the invention preferably consists of a K type CpG ODN, poly-dA, and any spacer sequence, and more preferably consists of a K type CpG ODN and poly-dA.

In the most preferred embodiment, the oligodeoxynucleotide of the invention consists of a K type CpG ODN (specific example: oligodeoxynucleotide consisting of a nucleotide sequence set forth in SEQ ID NO: 1) and poly-dA, and the K type CpG ODN is positioned at the 5′ end of the oligodeoxynucleotide and the poly-dA is positioned at the 3′ end. Specifically, the oligodeoxynucleotide of the invention is an oligodeoxynucleotide to which poly-dA that is 20 to 60 nucleotides long (more preferably 30 to 50 nucleotides long (30, 31, 32, 33, 34, 35, 36, 37, 38, 39, 40, 41, 42, 43, 44, 45, 46, 47, 48, 49, or 50 nucleotides long) and most preferably 30 to 45 nucleotides long (30, 31, 32, 33, 34, 35, 36, 37, 38, 39, 40, 41, 42, 43, 44, or 45 nucleotides long)) is bound to the 3′ end of an oligodeoxynucleotide consisting of the nucleotide sequence set forth in SEQ ID NO: 1. For example, it is an oligodeoxynucleotide consisting of the nucleotide sequence set forth in SEQ ID NO: 2 or 9 to 12.

The full length of the oligodeoxynucleotide of the invention is generally 30 to 200 nucleotides long, preferably 35 to 100 nucleotides long, more preferably 40 to 80 nucleotides long (specifically, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80 nucleotides long), more preferably 50 to 70 nucleotides long (specifically, 50, 51, 52, 53, 54, 55, 56, 57, 58, 59, 60, 61, 62, 63, 64, 65, 66, 67, 68, 69, or 70 nucleotides long), and most preferably 50 to 65 nucleotides long (specifically, 50, 51, 52, 53, 54, 55, 56, 57, 58, 59, 60, 61, 62, 63, 64, or 65 nucleotides long). The oligodeoxynucleotide of the invention may be suitably modified to be resistant to degradation in vivo (e.g., degradation due to exo- or endonuclease). Preferably, the modification comprises a phosphorothioate modification or phosphorodithioate modification. That is, some or all of phosphodiester bonds in the oligodeoxynucleotide of the invention are substituted with a phosphorothioate bond or a phosphorodithioate bond.

Preferably, the oligodeoxynucleotide of the invention comprises a phosphodiester bond, and more preferably the modification of a phosphodiester bond is a phosphorothioate bond (i.e., as described in WO 95/26204, one of the non-crosslinked atoms is substituted with a sulfur atom). That is, some or all of phosphodiester bonds are substituted with a phosphorothioate bond.

The oligodeoxynucleotide of the invention preferably comprises a modification with a phosphorothioate bond or a phosphorodithioate bond in a K type CpG ODN, and more preferably some or all of phosphodiester bonds of the K type CpG ODN are substituted with a phosphorothioate bond. Further, the oligodeoxynucleotide of the invention preferably comprises a phosphorothioate bond or a phosphorodithioate bond in poly-dA, and more preferably all phosphodiester bonds of the poly-dA are substituted with a phosphorothioate bond. Still more preferably, some or all of phosphodiester bonds of the oligodeoxynucleotide comprising a humanized K type CpG oligodeoxynucleotide and polydeoxyadenylic acid are substituted with a phosphorothioate bond. The most preferably, the oligodeoxynucleotide of the invention is an oligodeoxynucleotide to which poly-dA that is 20 to 60 nucleotides long (more preferably 30 to 50 nucleotides (30, 31, 32, 33, 34, 35, 36, 37, 38, 39, 40, 41, 42, 43, 44, 45, 46, 47, 48, 49, or 50 nucleotides long) and most preferably 30 to 45 nucleotides long (30, 31, 32, 33, 34, 35, 36, 37, 38, 39, 40, 41, 42, 43, 44, or 45 nucleotides long)) is bound to the 3′ end of a humanized K type CpG oligodeoxynucleotide (e.g., SEQ ID NO: 1), wherein all phosphodiester bonds comprised in the oligodeoxynucleotide are substituted with a phosphorothioate bond. This is because it is expected that a phosphorothioate bond results in not only resistance to degradation, but also enhanced immunostimulatory activity (e.g., activity to activate pDCs to induce IFN-α production), high yield of CpG-β-1,3-glucan complexes, and enhanced anticancer activity in the oligodeoxynucleotide of the invention. As used herein, a phosphorothioate bond is synonymous with a phosphorothioate backbone, and a phosphodiester bond is synonymous with a phosphoric acid backbone. The oligodeoxynucleotide of the invention includes all pharmaceutically accepted salts and esters or salts of such esters of the above-described oligodeoxynucleotide.

Preferred pharmaceutically acceptable salts of the oligodeoxynucleotide of the invention include alkali metal salts such as sodium salt, potassium salt, lithium salt; alkaline earth metal salts such as calcium salt and magnesium salt; metal salts such as aluminum salt, iron salt, zinc salt, copper salt, nickel salt, and cobalt salt; inorganic amine salts such as ammonium salts; organic amine salts such as t-octylamine salt, dibenzylamine salt, morpholine salt, glucosamine salt, phenylglycine alkyl ester salt, ethylenediamine salt, N-methylglucamine salt, guanidine salt, diethylamine salt, triethylamine salt, dicyclohexylamine salt, N,N′-dibenzylethylenediamine salt, chloroprocaine salt, procaine salt, diethanolamine salt, N-benzylphenethylamine salt, piperazine salt, tetramethylammonium salt, and tris(hydroxymethyl)aminomethane salt; hydrohalide salts such as hydrofluorate salt, hydrochloride salt, hydrobromide salt and hydroiodide salt; inorganic acid salts such as nitrates, perchlorates, sulfates, and phosphates; lower alkane sulfonates such as methanesulfonates, trifluoromethanesulfonates and ethanesulfonates; arylsulfonates such as benzenesulfonates and p-toluenesulfonates; organic acid salts such as acetates, malates, fumarates, succinates, citrates, tartarates, oxalates, and maleates; and, amino acid salts such as glycine salt, lysine salt, arginine salt, ornithine salt, glutamic acid salt and aspartic acid salt.

The oligodeoxynucleotide of the invention may have a single stranded, double stranded, or triple stranded form, but preferably has a single stranded form.

The oligodeoxynucleotide of the invention is preferably isolated. “Isolated” means that agents other than the component of interest are removed such that the substance is no longer in a naturally-occurring state. The purity of an “isolated oligodeoxynucleotide” (percentage of the weight of the oligodeoxynucleotide of interest accounting for the total weight of the evaluation target) is generally 70% or greater, preferably 80% or greater, more preferably 90% or greater, and still more preferably 99% or greater.

The oligodeoxynucleotide of the invention has excellent immunostimulatory activity (e.g., activity to activate B cells (preferably human B cells) to induce IL-6 production), so that it is useful as an immunostimulatory agent or the like. The oligodeoxynucleotide of the invention is useful in the preparation of the complex of the invention as it has a property of forming a triple helix structure with two β-1,3-glucans (preferably, schizophyllan, lentinan, or scleroglucan).

The present invention provides a complex comprising the above-described oligodeoxynucleotide of the invention and β-1,3-glucan (hereinafter, referred to as the complex of the invention).

The aforementioned oligodeoxynucleotide of the invention comprises a K type CpG ODN, such that this alone exerts immunostimulatory activity unique to K type CpG ODNs (e.g., activity to activate B cells (preferably human B cells) to induce IL-6 production), but lacks immunostimulatory activity unique to D type CpG ODNs (e.g. activity to activate plasmacytoid dendritic cells to induce IFN-α production). However, immunostimulatory activity unique to D type CpG ODNs (e.g. activity to activate plasmacytoid dendritic cells to induce IFN-α production) is surprisingly acquired without a sequence of a D type CpG ODN by forming a complex with β-1,3-glucan (preferably lentinan or schizophyllan). That is, the complex of the invention has both immunostimulatory activity unique to K type CpG ODNs (e.g., activity to activate B cells (preferably human B cells) to induce IL-6 production) and immunostimulatory activity unique to D type CpG ODNs (e.g. activity to activate plasmacytoid dendritic cells (preferably human plasmacytoid dendritic cells) to induce IFN-α production). Examples of β-1,3-glucan used in the present invention include schizophyllan, scleroglucan, curdlan, pachyman, grifolan, lentinan, laminaran, and the like. β-1,3-glucan preferably comprises many 1,6-glucopyranoside branches (side change ratio of 33-40%) as in schizophyllan, lentinan, or scleroglucan, and is more preferably schizophyllan.

Lentinan (LNT) is a known β-1,3-1,6-glucan derived from shiitake mushrooms. The molecular formula is (C6H10O5)n, and the molecular weight is about 300000 to 700000. Lentinan hardly dissolves in water, methanol, ethanol (95), or acetone, but dissolves in polar organic solvents, DMSO and aqueous sodium hydroxide solution.

Lentinan has action to enhance activated macrophage, killer T cell, natural killer cell, and antibody dependent macrophage mediated cytotoxicity (ADMC) activity (Hamuro, J., et al.: Immunology, 39, 551-559, 1980, Hamuro, J., et al.: Int. J. Immunopharmacol., 2, 171, 1980, Herlyn, D., et al.: Gann, 76, 37-42, 1985). In animal experiments, lentinan is recognized as having tumor growth suppressing action and a life prolongation effect by combined administration with a chemotherapeutic agent on isologous and autologous tumor. Lentinan is also recognized as having tumor growth suppressing action and a life prolongation effect in administration of lentinan alone. Lentinan is recognized as prolonging the survival period by combined use with tegafur oral administration on inoperable or recurrent gastric cancer patients in clinical trials (Interview form for “Lentinan I.V. infusion 1 mg ‘Ajinomoto’”), which is approved in Japan. The effect of administration of lentinan alone has not been confirmed.

Schizophyllan (SPG) is a known soluble β-glucan derived from Schizophyllum commune. SPG consists of a main chain of β-(1→3)-D-glucan and a β-(1→6)-D-glucosyl side chain for each three glucoses (Tabata, K., Ito, W., Kojima, T., Kawabata, S. and Misaki A., “Carbohydr. Res.”, 1981, 89, 1, p. 121-135). SPG has been used for over 20 years as an intramuscular injection formulation and clinical drug for gynecologic cancer (Shimizu, Chin, Hasumi, Masubuchi, “Biotherapy”, 1990, 4, p. 1390 Hasegawa, “Oncology and Chemotherapy”, 1992, 8, p. 225), such that the in vivo safety thereof has been confirmed (Theresa, M. McIntire and David, A. Brant, “J. Am. Chem. Soc.”, 1998, 120, p. 6909).

As used herein, “complex” refers to a product obtained by multiple molecules associating via a covalent bond or a non-covalent bond such as electrostatic bond, van der Waals bond, hydrogen bond, or hydrophobic interaction.

The complex of the invention is preferably in a triple helix structural form. In a preferred embodiment, two of three chains forming the triple helix structure are β-1,3-glucan chains and one is a chain of polydeoxyadenylic acid in the oligodeoxynucleotide of the invention described above. It should be noted that the complex may partially comprise a portion that does not form a triple helix structure.

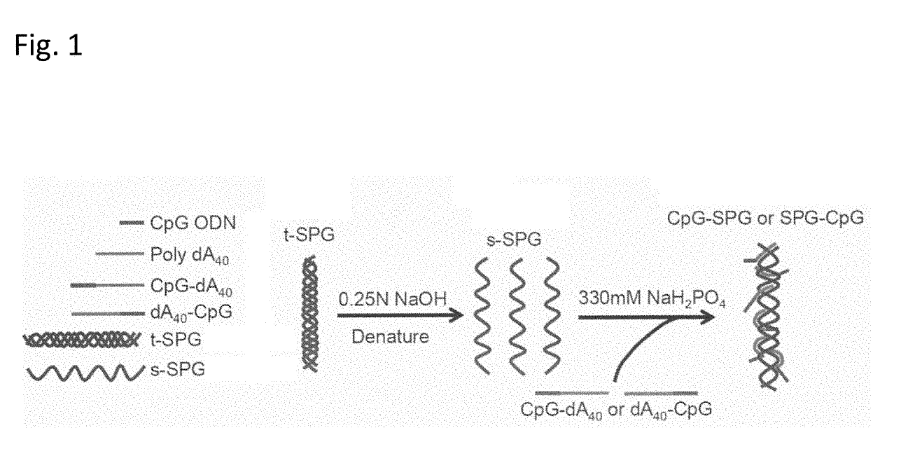

The composition ratio of the oligodeoxynucleotide and β-1,3-glucan in the complex of the invention may vary depending on the chain length of polydeoxyadenylic acid, the length of β-1,3-glucan, and the like in the oligodeoxynucleotide. For instance, when the β-1,3-glucan chain and the polydeoxyadenylic acid chain have the same length, two β-1,3-glucan chains may associate with one oligodeoxynucleotide of the invention to form a triple helix structure. In general, polydeoxyadenylic acid chains are shorter than β-1,3-glucan chains. Thus, multiple oligodeoxynucleotides of the invention may associate with two β-1,3-glucan chains via polydeoxyadenylic acid to form a triple helix structure (see FIG. 1).

The complex of the invention is a complex comprising a humanized K type CpG ODN and β-1,3-glucan (e.g., lentinan, schizophyllan, scleroglucan, curdlan, pachyman, grifolan, or laminaran), preferably a complex consisting of a humanized K type CpG ODN and β-1,3-glucan (e.g., lentinan, schizophyllan, or scleroglucan). More preferably, the complex of the invention is a complex consisting of β-1,3-glucan (e.g., lentinan or schizophyllan) and an oligodeoxynucleotide wherein polydeoxyadenylic acid that is 20 to 60 nucleotides long (specifically, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 nucleotides long) is bound to the 3′ side of an oligodeoxynucleotide consisting of the nucleotide sequence set forth in SEQ ID NO: 1 and all of phosphodiester bonds are substituted with a phosphorothioate bond (e.g., K3-dA20-60-LNT and K3-dA20-60-SPG), still more preferably a complex consisting of β-1,3-glucan (e.g., lentinan or schizophyllan) and an oligodeoxynucleotide wherein polydeoxyadenylic acid that is 30 to 50 nucleotides long (specifically, 30, 31, 32, 33, 34, 35, 36, 37, 38, 39, 40, 41, 42, 43, 44, 45, 46, 47, 48, 49, or 50 nucleotides long) is bound to the 3′ side of an oligodeoxynucleotide consisting of the nucleotide sequence set forth in SEQ ID NO: 1 and all of phosphodiester bonds are substituted with a phosphorothioate bond (e.g., K3-dA30-50-LNT and K3-dA30-50-SPG), and most preferably a complex consisting of 3-1,3-glucan (e.g., lentinan or schizophyllan) and an oligodeoxynucleotide wherein polydeoxyadenylic acid that is 30 to 45 nucleotides long (specifically, 30, 31, 32, 33, 34, 35, 36, 37, 38, 39, 40, 41, 42, 43, 44, or 45 nucleotides long) is bound to the 3′ side of an oligodeoxynucleotide consisting of the nucleotide sequence set forth in SEQ ID NO: 1 and all of phosphodiester bonds are substituted with a phosphorothioate bond (e.g., K3-dA30-45-LNT and K3-dA30-45-SPG).

A method of preparation of the complex of the invention can be carried out under the same conditions that are described in Non Patent Literatures 21 to 24 and Japanese Laid-Open Publication No. 2008-100919. That is, β-1,3-glucan which is naturally present as a triple helix structure is dissolved into aprotonic organic polar solvent (dimethyl sulfoxide (DMSO), acetonitrile, acetone or the like) to obtain a single chain. A solution of a single chain of β-1,3-glucan obtained in this manner is mixed with a solution of the oligodeoxynucleotide of the invention (aqueous solution, buffer solution with near neutral or acidic pH, preferably an aqueous solution or a buffer solution with near neutral pH), and pH is readjusted to near neutral as needed and then the mixture is maintained for a suitable amount of time, such as overnight, at 5° C. As a result, two β-1,3-glucan chains and poly-dA chain in the oligodeoxynucleotide form a triple helix structure, thus forming the complex of the invention. Oligodeoxynucleotide that fail to form a complex can be removed by purification using size exclusion chromatography, ultrafiltration, dialysis, or the like on the generate complexes. β-1,3-glucan that fails to form a complex can also be removed by purification using anion exchange chromatography on the generated complexes. Complexes can be appropriately purified by the above-described method.

Formation of the complex of the invention can be confirmed by measuring, for example, conformational change from CD (circular dichroism) spectra, UV absorption shift by size exclusion chromatography, gel electrophoresis, microchip electrophoresis, or capillary electrophoresis, but this is not limited thereto.

The mixing ratio of the oligodeoxynucleotide and β-1,3-glucan of the invention can be appropriately determined by considering the length of a poly-dA chain or the like, but the mole ratio (SPG/ODN) is generally 0.02 to 2.0 and preferably 0.1 to 0.5. In a further embodiment, the mole ratio (β-1,3-glucan (LNT or the like)/ODN) is for example, 0.005 to 1.0 and preferably 0.020 to 0.25.

The preparation method of the complex of the invention is explained with CpG-ODN and LNT complexes as an example. LNT is dissolved in 0.05 to 2 N, preferably 0.1 to 1.5 N aqueous alkali solution (e.g., 0.25 N aqueous sodium hydroxide solution), and the solution is left standing for 10 hours to 4 days at 1° C. to 40° C. (e.g., left standing overnight at room temperature) to prepare a single chain aqueous LNT solution (e.g., 50 mg/ml aqueous LNT solution). An aqueous CpG solution (e.g., 100 μM aqueous CpG solution) that was prepared separately and the aqueous LNT solution are mixed at a mole ratio (LNT/ODN) of 0.005 to 1.0, and an acidic aqueous buffer solution (e.g., NaH2PO4) is added to the aqueous LNT solution for neutralization. The mixture is maintained for 6 hours to 4 days at 1 to 40° C. (e.g., overnight at 4° C.) to complete the complex formation. It should be noted that the aqueous LNT solution may be added and mixed at the end for the complex formation. Complex formation can be confirmed, for example, by using size exclusion chromatography and monitoring absorption at 240 to 280 nm (e.g., 260 nm) for a shift to the high molecular weight side of CpG ODNs.

In one embodiment, the complex of the invention exhibits a rod-like particulate form. The particle size is the same as the size of particles of β-1,3-glucan (e.g., schizophyllan) used as the material naturally exhibiting a triple helix structure. The mean particle size is generally 10 to 100 nm and preferably 20 to 50 nm. The particle size can be measured by dissolving a complex in water and using dynamic light scattering under the condition of 80° C. with a Malvern Instruments Zeta Sizer.

The complex of the invention is preferably isolated. The purity of an “isolated complex” (percentage of the weight of the complex of interest accounting for the total weight of the evaluation target) is generally 70% or greater, preferably 80% or greater, more preferably 90% or greater, and still more preferably 99% or greater.

Furthermore, the complex of the invention has excellent immunostimulatory activity in addition to anticancer activity, especially both immunostimulatory activity unique to K type CpG ODNs (e.g., activity to activate B cells (preferably human B cells) to induce IL-6 production) and immunostimulatory activity unique to D type CpG ODNs (e.g. activity to activate plasmacytoid dendritic cells (preferably human plasmacytoid dendritic cells) to induce IFN-α production). Thus, the complex can be advantageous as it can also impart an effect as an immunostimulatory agent or the like. For instance, a complex comprising a K type CpG ODNs (e.g., SEQ ID NO: 2, 11, or 12) and SPG, and a complex comprising a K type CpG ODN (e.g., SEQ ID NO: 2) and SPG (K3-SPG) can be advantageous as a complex achieving inflammatory response induction capability (pan-IFN-α, IL-6 and the like), action to enhance serum antige-specific IgG antibody titer (Total IgG, IgG2c and the like) in virus inoculated individuals, antigen specific cytokine production capability (IFN-γ, IL2, and the like) in virus inoculated individuals, or protective effect against virus infections.

(Pharmaceutical Compositions)

The present invention provides a pharmaceutical composition comprising the above-described oligodeoxynucleotide of the invention or the above-described complex of the invention. The pharmaceutical composition of the invention can be obtained by formulating the above-described oligodeoxynucleotide of the invention or the above-described complex of the invention according to conventional means. The pharmaceutical composition of the invention comprises the oligodeoxynucleotide or complex of the invention and a pharmaceutically acceptable carrier. Further, the pharmaceutical composition of the invention may further comprise an antigen. Such a pharmaceutical composition is provided in a dosage form that is suitable for oral or parenteral administration.

Compositions for parenteral administration are used as, for example, injection, suppository, or the like, and injections may encompass dosage forms such as intravenous injection, subcutaneous injection, intradermal injection, intramuscular injection, and intravenous drip injection. Such an injection can be prepared according to a known method. For example, a method of preparing an injection can prepare an injection by dissolving or suspending the above-described oligodeoxynucleotide or complex of the invention in an aseptic aqueous solvent that is generally used in injections. Examples of aqueous solvents for injection that can be used include distilled water, saline, buffers such as phosphate buffer, carbonate buffer, tris buffer, and acetate buffer, and the like. The pH of such aqueous solvents can be 5 to 10, and preferably 6 to 8. Prepared injection solution is preferably filled in a suitable ampoule.

Further, a powdered formulation of the oligodeoxynucleotide or complex of the invention can be prepared by subjecting a suspension of the oligodeoxynucleotide or complex of the invention to vacuum drying, lyophilization or the like. The oligodeoxynucleotide or complex of the invention can be stored in a powdered state, and used by dispersing the powder in an aqueous solvent for injection upon use.

The content of the oligodeoxynucleotide or complex of the invention in a pharmaceutical composition is generally about 0.1 to 100% by weight, preferably about 1 to 99% by weight, and more preferably about 10 to 90% by weight of the entire pharmaceutical composition.

The pharmaceutical composition of the present invention may contain, as an effective ingredient, the oligodeoxynucleotide or complex of the invention alone, or the oligodeoxynucleotide or complex of the invention in combination with another effective ingredient.

(Pharmaceutical Application)

It has been discovered that the oligodeoxynucleotide and complex of the invention alone have anticancer action. Such an effect was unexpected from the characteristic of the present invention which has been developed as an adjuvant agent. Thus, the present invention provides an anticancer agent, which does not require the conventional usage as an adjuvant, i.e., administration with a cancer antigen, and which acts mildly to the body as a versatile anticancer agent that is not limited to a specific cancer type. The oligodeoxynucleotide and complex of the invention also has immunostimulatory activity. Thus, there is expectation for immunostimulatory activity against other diseases and synergistic effects on physically weakened cancer patients.

Since the present invention has excellent immunostimulatory activity in addition to anticancer action, the oligodeoxynucleotide, complex, and pharmaceutical composition of the invention can be used as an immunostimulatory agent. The oligodeoxynucleotide, complex, or pharmaceutical composition of the invention can be administered to mammals (primates such as humans, rodents such as mice, and the like) to elicit an immune response in the mammals. In particular, the complex of the invention has a characteristic of activity of D type CpG ODNs, stimulating peripheral blood mononuclear cells to induce production of a large quantity of both type I interferon (Pan-IFN-α, IFN-α2, and the like) and type II interferon (IFN-γ). Thus, the complex is useful as a type I interferon production inducing agent, type II interferon production inducing agent, or type I and type II interferon production inducing agent. Since the production of both type I and type II interferons is induced, the complex of the invention and pharmaceutical compositions comprising the same are useful in the prevention of therapy of diseases in which one or both of type I and type II interferons is effective.

As an example of a method of materializing the pharmaceutical application, (a) a composition comprising the oligodeoxynucleotide of the invention or the complex of the invention can be administered to a cancer patient or a human who may have cancer to antigen-specifically activate cytotoxic T lymphocytes (CTL) in the subject who received the administration to directly prevent/treat the cancer (as an effect as a monotherapy).

As used herein, “subject” refers to a target subjected to the diagnosis, detection, therapy, or the like of the present invention (e.g., organisms such as humans, or cells, blood, serum, or the like extracted from an organism).

As used herein, “agent” is broadly used and may be any substance or other element (e.g., light, radiation, heat, electricity, and other forms of energy) as long as the intended objective can be achieved. Examples of such a substance include, but are not limited to, proteins, polypeptides, oligopeptides, peptides, polynucleotides, oligonucleotides, nucleotides, nucleic acids (including for example DNAs such as cDNAs and genomic DNAs and RNAs such as mRNAs), polysaccharides, oligosaccharides, lipids, organic small molecules (e.g., hormones, ligands, information transmitting substances, organic small molecules, molecules synthesized by combinatorial chemistry, small molecules that can be used as medicine (e.g., small molecule ligands and the like) and the like) and a composite molecules thereof. Typical examples of an agent specific to a polynucleotide include, but are not limited to, polynucleotides with complementarity to a certain sequence homology (e.g., 70% or greater sequence identity) to a sequence of the polynucleotide, polypeptides such as transcription factors that bind to a promoter region, and the like. Typical examples of an agent specific to a polypeptide include, but are not limited to, antibodies directed specifically to the polypeptide or derivatives or analogs thereof (e.g., single chain antibody), specific ligands or receptors when the polypeptide is a receptor or ligand, substrates thereof when the polypeptide is an enzyme, and the like.

As used herein, “therapy” refers to the prevention of exacerbation, preferably maintaining the current condition, more preferably alleviation, and still more preferably elimination of a disease or disorder (e.g., cancer or allergy) in case of such a condition, including being capable of exerting a an effect of improving or preventing a disease in a patient, or one or more symptoms accompanying the disease. Preliminary diagnosis conducted for suitable therapy may be referred to as a “companion therapy”, and a diagnostic drug therefor may be referred to as “companion diagnostic drug”.

As used herein, “therapeutic agent” broadly refers to all agents that are capable of treating a condition of interest (e.g., diseases such as cancer or allergy). In one embodiment of the present invention, “therapeutic drug” may be a pharmaceutical composition comprising an effective ingredient, and one or more pharmacologically acceptable carriers. A pharmaceutical composition can be manufactured, for example, by mixing an effective ingredient and the above-described carriers, by any method known in the technical field of pharmaceuticals. Further, the usage form of a therapeutic drug is not limited, as long as it is used for therapy. A therapeutic agent may consist solely of an effective ingredient or may be a mixture of an effective ingredient and any ingredient. Further, the shape of the above-described carriers is not particularly limited. For example, the carrier may be a solid or liquid (e.g., buffer). Therapeutic drugs for cancer or allergies include drugs (prophylactic drug) used for the prevention of cancer, allergies, or the like, and suppressants of cancer, allergies, or the like.

As used herein, “prevention” refers to the act of taking a measure against a disease or disorder (e.g., allergy) from being in such a condition, prior to the onset of such a condition. For example, it is possible to use the agent of the invention to perform diagnosis, and use the agent of the invention, as needed, to prevent or take measures to prevent allergies or the like.

As used herein, “prophylactic drug (agent)” broadly refers to all agents that are capable of preventing a condition of interest (e.g., disease such as an allergy or the like).

As used herein, “kit” refers to a unit providing portions to be provided (e.g., testing drug, diagnostic drug, therapeutic drug, antibody, label, manual, and the like), generally in two or more separate sections. This form of a kit is preferred when a composition that should not be provided in a mixed state is preferably mixed immediately before use for safety reasons or the like, is intended to be provided. Such a kit advantageously comprises instruction or manual preferably describing how the provided portions (e.g., testing drug, diagnostic drug, or therapeutic drug) should be used or how a reagent should be handled. When the kit is used herein as a reagent kit, the kit generally comprises an instruction describing how to use a testing drug, diagnostic drug, therapeutic drug, antibody, and the like.

As used herein, “instruction” is a document with an explanation of the method of use of the present invention for a physician or for other users. The instruction describes a detection method of the present invention, how to use a diagnostic drug, or a description instructing administration of a medicament or the like. Further, an instruction may have a description instructing oral administration, or administration to the esophagus (e.g., by injection or the like) as the site of administration. The instruction is prepared in accordance with a format defined by a regulatory authority of the country in which the present invention is practiced (e.g., Health, Labor and Welfare Ministry in Japan, Food and Drug Administration (FDA) in the U.S. or the like), with an explicit description showing approval by the regulatory authority. The instruction is a so-called package insert and is generally provided in, but not limited to, paper media. The instructions may also be provided in a form such as electronic media (e.g., web sites provided on the Internet or emails).

### Preferred Embodiments

The preferred embodiments of the present invention are explained hereinafter. It is understood that the embodiments provided hereinafter are provided to better facilitate the understanding of the present invention, so that the scope of the present invention should not be limited by the following description. Thus, it is apparent that those skilled in the art can refer to the descriptions herein to appropriately make modifications within the scope of the present invention. It is also understood that the following embodiments of the present invention can be used individually or as a combination.

### <Monotherapeutic Form>

In one embodiment, the present invention provides an anticancer agent comprising a complex, comprising: (a) an oligodeoxynucleotide comprising a humanized K type CpG oligodeoxynucleotide and polydeoxyadenylic acid, wherein the polydeoxyadenylic acid is disposed on the 3′ side of the humanized K type CpG oligodeoxynucleotide; and (b) β-1,3-glucan. The present invention has discovered that the complex of the invention itself acts as an anticancer agent. The inventors previously only discovered that the complex can be used as an adjuvant and filed for a patent. It was unexpected that the complex can be used directly as an anticancer agent as a monotherapy. Thus, the complex of the invention achieves an unexpected effect in terms of its use without cancer antigens.

In one embodiment, the anticancer agent of the invention is administered without a cancer antigen.

In another embodiment, the anticancer agent of the invention is administered to be delivered to a reticuloendothelial system and/or a lymph node. Preferably, the reticuloendothelial system and/or lymph node comprises tumor and phagocytes. For example, the reticuloendothelial system and/or lymph node comprises a spleen and/or a liver. Thus, the anticancer agent of the invention is administered to be delivered to a reticuloendothelial system organ (spleen, liver, or the like) and/or a lymph node comprising tumor and phagocytes. Although not wishing to be bound by any theory, it has been shown that the complex of the invention is delivered to tumor and phagocytes, where dead cancer cells are recruited to the reticuloendothelial system organ (spleen, liver, or the like). It is understood that cancer cells within the body can be further eliminated thereby. Thus, the present invention is recognized as achieving a significant effect in terms of being able to provide an anticancer agent that did not exist previously, which is not an adjuvant for a specific cancer using a specific cancer antigen but can kill any cancer that is present in the body.

Thus, in a more preferred embodiment, the anticancer agent of the invention is administered to be delivered to tumor and phagocytes without a cancer antigen.

Any method can be used as such a delivery method. For example, the administration includes, but is not limited to, systemic administration. The administration is preferably systemic administration. Examples of systemic administration include intravenous administration, intraperitoneal administration, oral administration, subcutaneous administration, intramuscular administration, and the like.

In one embodiment, the oligodeoxynucleotide used in the present invention include, but are not limited to, K3 (SEQ ID NO: 1), K3-dA₄₀ (SEQ ID NO: 2), dA₄₀-K3 (SEQ ID NO: 3), K3-dA20 (SEQ ID NO: 4), K3-dA25 (SEQ ID NO: 5), K3-dA30 (SEQ ID NO: 6), K3-dA35 (SEQ ID NO: 7), and the like.

In one embodiment, the β-1,3-glucan used in the present invention may be schizophyllan (SPG), lentinan, scleroglucan, curdlan, pachyman, grifolan, laminaran, or the like.

In a preferred embodiment, the complex of the invention is K3-SPG or an analog thereof. In this regard, examples of analogs include, but are not limited to, complexes with a structure similar to K3 on the CpG side, complexes with a structure similar to SPG on the β-glucan side, and the like.

Since anticancer action involves various mechanisms, applications for inducing accumulation of dead cancer cells in the spleen or the like are not readily conceived. In particular, applications for inducing accumulation of tumor cells that have accumulated and died in tumor in tissue such as the spleen in systemic administration is not conceivable. Further, expression of interleukin 12 (IL12) and/or interferon (IFN)α and an effect of enhancing the same involve mechanisms that are different from anticancer action. In addition, expression of interleukin 12 (IL12) and/or interferon (IFN)α and an effect of enhancing the same can be exerted by action other than anticancer. Thus, they are not readily conceived from each other. Hence, each of the applications of the CpG-p glucan complex of the invention (anticancer application (as a monotherapy), application for inducing accumulation of dead cancer cells in the spleen, and expression of interleukin 12 (IL12) and/or interferon (IFN)α and the enhancement thereof) are not related so that they can be readily conceived from each other.

<Agent Inducing Accumulation in Reticuloendothelial System (Including the Spleen and/or Liver) and/or Lymph Node>

In another aspect, the present invention provides a composition for inducing accumulation of dead cancer cells in a reticuloendothelial system (including the spleen and/or a liver, comprising a complex comprising: (a) an oligodeoxynucleotide comprising a humanized K type CpG oligodeoxynucleotide and polydeoxyadenylic acid, wherein the polydeoxyadenylic acid is disposed on the 3′ side of the humanized K type CpG oligodeoxynucleotide; and (b) β-1,3-glucan. Although not wishing to be bound by any theory, it has been discovered that the complex of the invention can induce accumulation of dead cancer cells in a reticuloendothelial system (including the spleen and/or liver) and/or lymph node. The Examples demonstrate that treatment with the complex of the invention such as K3-SPG induces tumor cell death in a manner dependent on both IL12p40 and IFN-I. It was previously unexpected that a complex had such an action. In this context, an unexpected working effect is achieved. That is, CpG is targeted by phagocytes in a tumor microenvironment. When dead cancer cells accumulate in a reticuloendothelial system (including the spleen and/or liver) and/or lymph node, released dead tumor cells subsequently induce antitumor CTL to multiple tumor antigens, such that cancer cells in the body can be killed as if they are attacked with a shot gun and eradicated. Although not wishing to be bound by any theory, production of both IL12 and IFN-I cytokines in a tumor microenvironment cannot be considered essential, but is important for K3-SPG monotherapy.

In one embodiment, the oligodeoxynucleotide used in the present invention is selected from the group consisting of K3 (SEQ ID NO: 1), K3-dA₄₀ (SEQ ID NO: 2), dA₄₀-K3 (SEQ ID NO: 3), K3-dA20 (SEQ ID NO: 4), K3-dA25 (SEQ ID NO: 5), K3-dA30 (SEQ ID NO: 6), and K3-dA35 (SEQ ID NO: 7).

In another embodiment, the β-1,3-glucan used in the present invention is selected from the group consisting of schizophyllan (SPG), scleroglucan, curdlan, pachyman, grifolan, and laminaran.

In a preferred embodiment, the complex of the invention is K3-SPG.

In one embodiment, the reticuloendothelial system and/or lymph node targeted by the composition of the invention comprises tumor and phagocytes. For example, the reticuloendothelial system comprises a spleen and/or a liver. Thus, the composition of the invention is administered to be delivered to a reticuloendothelial system organ (spleen, liver, or the like) and/or a lymph node comprising tumor and phagocytes. Although not wishing to be bound by any theory, it has been shown that the complex of the invention is delivered to tumor and phagocytes, where dead cancer cells are recruited to the reticuloendothelial system organ (spleen, liver, or the like). It is understood that cancer cells within the body can be further eliminated thereby. Thus, the present invention is recognized as achieving a significant effect in terms of being able to provide an anticancer agent that did not exist previously, which is not an adjuvant for a specific cancer using a specific cancer antigen but can kill any cancer that is present in the body.

Thus, in a more preferred embodiment, the anticancer agent of the invention is administered to be delivered to tumor and phagocytes without a cancer antigen.

Any method can be used as such a delivery method. For example, the administration includes, but is not limited to, systemic administration. The administration is preferably systemic administration. Examples of systemic administration include intravenous administration, intraperitoneal administration, oral administration, subcutaneous administration, intramuscular administration, and the like.

<IL12 and/or IFN Expression Enhancing Agent>

In still another aspect, the present invention provides a composition for the expression of interleukin 12 (IL12) and/or interferon (IFN)γ or the enhancement thereof, comprising: (a) an oligodeoxynucleotide comprising a humanized K type CpG oligodeoxynucleotide and polydeoxyadenylic acid, wherein the polydeoxyadenylic acid is disposed on the 3′ side of the humanized K type CpG oligodeoxynucleotide; and (b) β-1,3-glucan. Production of both IL12 and IFN-I cytokines in a tumor microenvironment is an important working effect in a K3-SPG monotherapy. Such an effect is important for action as an anticancer agent as well as for other applications. Subjects of such treatment include, but are not limited to, cancer, chronic infectious diseases of a virus or the like, viral infection prevention, and the like.

In one embodiment, the oligodeoxynucleotide used in the present invention is selected from the group consisting of K3 (SEQ ID NO: 1), K3-dA₄₀ (SEQ ID NO: 2), dA₄₀-K3 (SEQ ID NO: 3), K3-dA20 (SEQ ID NO: 4), K3-dA25 (SEQ ID NO: 5), K3-dA30 (SEQ ID NO: 6), and K3-dA35 (SEQ ID NO: 7).

In another embodiment, the β-1,3-glucan used in the present invention is selected from the group consisting of schizophyllan (SPG), scleroglucan, curdlan, pachyman, grifolan, and laminaran.

In a preferred embodiment, the complex of the invention is K3-SPG.

(Medicament, Dosage Form, Etc.)

The present invention is provided as a medicament (therapeutic drug or prophylactic drug) in various forms described above.

The route of administration of a therapeutic drug that is effective upon therapy is preferably used, such as intravenous, subcutaneous, intramuscular, intraperitoneal, or oral administration, or the like. Examples of dosage forms include injections, capsules, tablets, granules, and the like. The components of the present invention are effectively used upon administration as an injection. Aqueous solutions for injection may be stored, for example, in a vial or a stainless steel container. Aqueous solutions for injections may also be blended with, for example, saline, sugar (e.g., trehalose), NaCl, NaOH, or the like. Therapeutic drugs may also be blended, for example, with a buffer (e.g., phosphate buffer), stabilizer, or the like.

In general, the composition, medicament, therapeutic agent, prophylactic agent, or the like of the present invention comprises a therapeutically effective amount of a therapeutic agent or effective ingredient, and a pharmaceutically acceptable carrier or excipient. As used herein, “pharmaceutically acceptable” means that a substance is approved by a government regulatory agency or listed in the pharmacopoeia or other commonly recognized pharmacopoeia for use in animals, more specifically in humans. As used herein, “carrier” refers to a diluent, adjuvant, excipient or vehicle that is administered with a therapeutic agent. Such a carrier can be an aseptic liquid such as water or oil, including, but not limited to, those derived from petroleum, animal, plant, or synthesis, as well as peanut oil, soybean oil, mineral oil, sesame oil, and the like. When a medicament is orally administered, water is a preferred carrier. For intravenous administration of a pharmaceutical composition, saline and aqueous dextrose are preferred carriers. Preferably, an aqueous saline solution and aqueous dextrose and glycerol solution are used as a liquid carrier of an injectable solution. Suitable excipients include light anhydrous silicic acid, crystalline cellulose, mannitol, starch, glucose, lactose, sucrose, gelatin, malt, rice, wheat flour, chalk, silica gel, sodium stearate, glyceryl monostearate, talc, sodium chloride, powdered skim milk, glycerol, propylene, glycol, water, ethanol, carmellose calcium, carmellose sodium, hydroxypropyl cellulose, hydroxypropyl methylcellulose, polyvinyl acetal diethylamino acetate, polyvinylpyrrolidone, gelatin, medium-chain fatty acid triglyceride, polyoxyethylene hydrogenated castor oil 60, saccharose, carboxymethylcellulose, corn starch, inorganic salt, and the like. When desirable, the composition can also contain a small amount of wetting agent, emulsifier, or pH buffer. These compositions can be in a form of a solution, suspension, emulsion, tablet, pill, capsule, powder, sustained release preparation, or the like. It is also possible to use traditional binding agents and carriers, such as triglyceride, to prepare a composition as a suppository. Oral preparation can also comprise a standard carrier such as medicine grade mannitol, lactose, starch, magnesium stearate, sodium saccharin, cellulose, or magnesium carbonate. Examples of a suitable carrier are described in E. W. Martin, Remington's Pharmaceutical Sciences (Mark Publishing Company, Easton, U.S.A.). Such a composition contains a therapeutically effective amount of therapy agent, preferably in a purified form, together with a suitable amount of carrier, such that the composition is provided in a form that is suitable for administration to a patient. A preparation must be suitable for the administration form. In addition, the composition may comprise, for example, a surfactant, excipient, coloring agent, flavoring agent, preservative, stabilizer, buffer, suspension, isotonizing agent, binding agent, disintegrant, lubricant, fluidity improving agent, corrigent, or the like.

Examples of “salt”, in one embodiment of the present invention, include anionic salts formed with any acidic (e.g., carboxyl) group and cationic salts formed with any basic (e.g., amino) group. Salts include inorganic salts and organic salts, as well as salts described in, for example, Berge et al., J. Pharm. Sci., 1977, 66, 1-19. Examples thereof further include metal salts, ammonium salts, salts with an organic base, salts with an inorganic acid, salts with an organic acid, and the like. “Solvate” in one embodiment of the present invention is a compound formed with a solute or solvent. For example, J. Honig et al., The Van Nostrand Chemist's Dictionary P650 (1953) can be referred to for solvates. When the solvent is water, a solvate formed thereof is a hydrate. It is preferable that the solvent does not obstruct the biological activity of the solute. Examples of such a preferred solvent include, but not particularly limited to, water and various buffers. Examples of “chemical modification” in one embodiment of the present invention include modifications with PEG or a derivative thereof, fluorescein modification, biotin modification, and the like.

Various delivery systems are known for administration of the present invention as a medicament. Such systems can be used to administer the therapeutic agent of the invention to a suitable site (e.g., esophagus). Examples of such a system include use of a recombinant cell that can express encapsulated therapeutic agent (e.g., polypeptide) in liposomes, microparticles, and microcapsules; use of endocytosis mediated by a receptor; construction of a therapy nucleic acid as a part of a retrovirus vector or another vector; and the like. Examples of the method of introduction include, but are not limited to, intradermal, intramuscular, intraperitoneal, intravenous, subcutaneous, intranasal, epidural, and oral routes. A medicament can be administered by any suitable route, such as by injection, by bolus injection, or by absorption through epithelia or mucocutaneous lining (e.g., oral cavity, rectum, intestinal mucosa, or the like). In addition, an inhaler or mistifier using an aerosolizing agent can be used as needed. Moreover, other biological activating agents can also be administered concomitantly. Administration can be systemic or local. When the present invention is used for cancer, the present invention can be administered by any suitable route such as a direct injection into cancer (lesion).

In a preferred embodiment, a composition can be prepared as a pharmaceutical composition adapted to administration to humans in accordance with a known method. Such a composition can be administered by an injection. A composition for injection is typically a solution in an aseptic isotonic aqueous buffer. A composition can also comprise a local anesthetic such as lidocaine, which alleviates the pain at the site of injection, and a solubilizing agent as needed. Generally, ingredients can be supplied individually or by mixing the ingredients together in a unit dosage form, and supplied, for example, in a sealed container such as an ampoule or sachet showing the amount of active agent, or as a lyophilized powder or water-free concentrate. When a composition is to be administered by injection, the composition can be distributed using an injection bottle containing aseptic agent-grade water or saline. When a composition is to be administered by injection, an aseptic water or saline ampoule for injection can also be provided such that the ingredients can be mixed prior to administration.

The composition, medicament, therapeutic agent, and prophylactic agent of the invention can be prepared with a neutral or base form or other prodrugs (e.g., ester or the like). Pharmaceutically acceptable salts include salts formed with a free carboxyl group, derived from hydrochloric acid, phosphoric acid, acetic acid, oxalic acid, tartaric acid, or the like, salts formed with a free amine group, derived from isopropylamine, triethylamine, 2-ethylaminoethanol, histidine, procaine, or the like, and salts derived from sodium, potassium, ammonium, calcium, ferric hydroxide or the like.

The amount of therapeutic agent of the invention that is effective in therapy of a specific disorder or condition may vary depending on the nature of the disorder or condition. However, such an amount can be determined by those skilled in the art with a standard clinical technique based on the descriptions herein. Furthermore, an in vitro assay can be used in some cases to assist the identification of the optimal dosing range. The precise dose to be used for a preparation may also vary depending on the route of administration or the severity of the disease or disorder. Thus, the dose should be determined in accordance with the judgment of the attending physician or the condition of each patient. The dosage is not particularly limited, but may be, for example, 0.001, 1, 5, 10, 15, 100, or 1000 mg/kg body weight per dose or within a range between any two values described above. The dosing interval is not particularly limited, but may be, for example, 1 or 2 doses every 1, 7, 14, 21, or 28 days, or 1 or 2 doses in a range of period between any two values described above. The dosage, dosing interval, and dosing method may be appropriately selected depending on the age, weight, symptom, target organ, or the like of the patient. Further, it is preferable that a therapeutic agent contains a therapeutically effective amount of effective ingredients, or an amount of effective ingredients effective for exerting a desired effect. When a malignant tumor marker significantly decreases after administration, presence of a therapeutic effect may be acknowledged. The effective dose can be estimated from a dose-response curve obtained from in vitro or animal model testing systems.

“Patient” or “subject” in one embodiment of the present invention includes humans and mammals excluding humans (e.g., one or more of mice, guinea pigs, hamsters, rats, rabbits, pigs, sheep, goats, cows, horses, cats, dogs, marmosets, monkeys, chimpanzees and the like).

The pharmaceutical composition, therapeutic agent, or prophylactic agent of the invention can be provided as a kit.

In a specific embodiment, the present invention provides an agent pack or kit comprising one or more containers filled with one or more ingredients of the composition or medicament of the invention. Optionally, information indicating approval for manufacture, use, or sale for administration to a human by a government agency regulating the manufacture, use, or sale of medicaments or biological products can be appended to such a container in a stipulated form.

In a specific embodiment, the pharmaceutical composition comprising an ingredient of the present invention can be administered via liposomes, microparticles, or microcapsules. In various embodiments of the present invention, it may be useful to use such a composition to achieve sustained release of the ingredient of the present invention.

The formulation procedure for the therapeutic drug, prophylactic drug, or the like of the invention as a medicament or the like is known in the art. The procedure is described, for example, in the Japanese Pharmacopoeia, the United States Pharmacopeia, pharmacopeia of other countries, or the like. Thus, those skilled in the art can determine the embodiment such as the amount to be used without undue experimentation from the descriptions herein.

(General Techniques)

Molecular biological approaches, biochemical approaches, and microbiological approaches used herein are well known and conventional approaches in the art that are described in, for example, Sambrook J. et al. (1989). Molecular Cloning: A Laboratory Manual, Cold Spring Harbor and its 3rd Ed. (2001); Ausubel, F. M. (1987). Current Protocols in Molecular Biology, Greene Pub. Associates and Wiley-Interscience; Ausubel, F. M. (1989). Short Protocols in Molecular Biology: A Compendium of Methods from Current Protocols in Molecular Biology, Greene Pub. Associates and Wiley-Interscience; Innis, M. A. (1990). PCR Protocols: A Guide to Methods and Applications, Academic Press; Ausubel, F. M. (1992). Short Protocols in Molecular Biology: A Compendium of Methods from Current Protocols in Molecular Biology, Greene Pub. Associates; Ausubel, F. M. (1995). Short Protocols in Molecular Biology: A Compendium of Methods from Current Protocols in Molecular Biology, Greene Pub. Associates; Innis, M. A. et al. (1995). PCR Strategies, Academic Press; Ausubel, F. M. (1999). Short Protocols in Molecular Biology: A Compendium of Methods from Current Protocols in Molecular Biology, Wiley, and annual updates; Sninsky, J. J. et al. (1999). PCR Applications: Protocols for Functional Genomics, Academic Press, Bessatsu Jikken Igaku [Experimental Medicine, Supplemental Volume], Idenshi Donyu Oyobi Hatsugen Kaiseki Jikken Ho [Experimental Methods for Transgenesis & Expression Analysis], Yodosha, 1997, and the like. The relevant portions (which can be the entire document) of the above documents are incorporated herein by reference.

DNA synthesis techniques and nucleic acid chemistry for making an artificially synthesized gene are described in, for example, Gait, M. J. (1985). Oligonucleotide Synthesis: A Practical Approach, IRL Press; Gait, M. J. (1990). Oligonucleotide Synthesis: A Practical Approach, IRL Press; Eckstein, F. (1991). Oligonucleotides and Analogues: A Practical Approach, IRL Press; Adams, R. L. et al. (1992). The Biochemistry of the Nucleic Acids, Chapman & Hall; Shabarova, Z. et al. (1994). Advanced Organic Chemistry of Nucleic Acids, Weinheim; Blackburn, G. M. et al. (1996). Nucleic Acids in Chemistry and Biology, Oxford University Press; Hermanson, G. T. (1996). Bioconjugate Techniques, Academic Press, and the like, the relevant portions of which are incorporated herein by reference.

For example, as used herein, the oligonucleotide of the invention can also be synthesized by a standard method known in the art, such as using an automated DNA synthesizer (a synthesizer commercially available from Biosearch, Applied Biosystems, or the like). For example, a phosphorothioate-oligonucleotide can also be synthesized by the method of Stein et al. (Stein et al., 1988, Nucl. Acids Res. 16: 3209), and a methyl phosphonate-oligonucleotide can also be prepared using a controlled pore glass polymer support (Sarin et al., 1988, Proc. Natl. Acad. Sci. USA 85: 7448-7451).

As used herein, “or” is used when “at least one or more” of the listed matters in the sentence can be employed. When explicitly described herein as “within the range of two values”, the range also includes the two values themselves.

Reference literatures such as scientific literatures, patents, and patent applications cited herein are incorporated herein by reference to the same extent that the entirety of each document is specifically described.

As described above, the present invention has been described while showing preferred embodiments to facilitate understanding. The present invention is described hereinafter based on Examples. The aforementioned description and the following Examples are not provided to limit the present invention, but for the sole purpose of exemplification. Thus, the scope of the present invention is not limited to the embodiments and Examples specifically described herein and is limited only by the scope of claims.

### EXAMPLES

The Examples are described hereinafter. When necessary, animals used in the following Examples were handled in compliance with the institutional guidelines set forth by the National Institute of Biomedical Innovation and the Institute of Experimental Animal Sciences of Osaka University, as well as the Declaration of Helsinki. For reagents, the specific products described in the Examples were used. However, the reagents can be substituted with an equivalent product from another manufacturer (Sigma-Aldrich, Wako Pure Chemical, Nacalai Tesque, R & D Systems, USCN Life Science INC, or the like).

### Manufacturing Examples

The following CpG ODNs were synthesized by GeneDesign, Inc. (underlines indicate phosphorothioate bonds).

TABLE 1
K3 (5′-ATC GAC TCT CGA GCG TTC TC-3′)
(SEQ ID NO: 1);
K3-dA₄₀ (5′-ATC GAC TCT CGA GCG TTC TC-40 mer A-3′)
(SEQ ID NO: 2);
dA₄₀-K3 (5′-40 mer A-ATC GAC TCT CGA GCG TTC TC-3′)
(SEQ ID NO: 3);
Alexa 488-labeled K3;
Alexa 488-labeled K3-dA₄₀;
Alexa 647-labeled K3;
Alexa 647-labeled K3-dA₄₀

In particular, the synthesis of K3-dA35 (SEQ ID NO: 7), K3-dA30 (SEQ ID NO: 6), K3-dA25 (SEQ ID NO: 5), and K3-dA20 (SEQ ID NO: 4) in addition to the above-described K3-dA40 (SEQ ID NO: 2) (Table 2) is described.

TABLE 2
K3-dA40: AsTsCsGsAsCsTsCsTsCsGsAsGsCsGsTsTsCsTsCs
AsAsAsAsAsAsAsAsAsAsAsAsAsAsAsAsAsAsAsAsAsAsAsAsAs
AsAsAsAsAsAsAsAsAsAsAsAsAsA (SEQ ID NO: 2 in
Sequence List)
K3-dA35: AsTsCsGsAsCsTsCsTsCsGsAsGsCsGsTsTsCsTsCs
AsAsAsAsAsAsAsAsAsAsAsAsAsAsAsAsAsAsAsAsAsAsAsAsAs
AsAsAsAsAsAsAsAsAsA (SEQ ID NO: 7 in Sequence
List)
K3-dA30: AsTsCsGsAsCsTsCsTsCsGsAsGsCsGsTsTsCsTsCs
AsAsAsAsAsAsAsAsAsAsAsAsAsAsAsAsAsAsAsAsAsAsAsAsAs
AsAsAsAsA (SEQ ID NO: 6 in Sequence List)
K3-dA25: AsTsCsGsAsCsTsCsTsCsGsAsGsCsGsTsTsCsTsCs
AsAsAsAsAsAsAsAsAsAsAsAsAsAsAsAsAsAsAsAsAsAsAsAsA
(SEQ ID NO: 5 in Sequence List)
K3-dA20: AsTsCsGsAsCsTsCsTsCsGsAsGsCsGsTsTsCsTsCs
AsAsAsAsAsAsAsAsAsAsAsAsAsAsAsAsAsAsAsA (SEQ ID
NO: 4 in Sequence List)

(s in the above-described sequences indicates that a phosphodiester bond between nucleosides is substituted with a phosphorothioate bond.)

The oligodeoxynucleotide was synthesized using a routine method, solid phase phosphoramidite method (Nucleic Acids in Chemistry and Biology, 3. Chemical synthesis (1990) ed. G. Michael Blackburn and Michael J. Gait. Oxford University Press).

Ovoalbumin (OVA) was purchased from Seikagaku Corporation. DQ-OVA, Alexa 488-OVA, CFSE, and Lipofectamine 2000 were purchased from Invitrogen. Hoechst 33258, zymosan, and curdlan were purchased from SIGMA. Zymosan-Depleted was purchased from Invivogen. Clodronate liposomes were purchased from FormuMax. Influenza split product vaccine, formalin inactivated whole virus (WIV), and purified influenza virus (H1N1) were prepared as previously described (Koyama, S., et al., Science translational medicine 2, 25ra24 (2010)).

Complex formation of CpG ODN and SPG (Manufacturing Example FIG. 1)

7.22 mg of K3-dA40 was dissolved in water (3.7 mL). 15 mg of SPG (Mitsui Sugar) was dissolved in 0.25 N NaOH (1 mL). 1 mL of 330 mM NaH2PO4 was added to a DNA solution, then the SPG solution was added to the DNA/NaH2PO4 solution. The mixture was maintained overnight at 4° C. to complete the complex formation. The mole ratio (MSPG/MDNA) was fixed at 0.27. Complex formation was confirmed with a MultiNA Microchip Electrophoresis System (SHIMADZU: MultiNA) by monitoring the absorption at 260 nm for a shift to the high molecular weight side of CpG ODNs using size exclusion chromatography (System: Agilent 1100 series, Column: Asahipak GF7M-HQ (Shodex) two linked columns, Flow rate: 0.8 mL/min, Buffer: 10 mM EDTA PBS, pH7.4, Temperature: 40° C.).

(Preparation for Use in Examples)

The following Examples demonstrated that systemic monotherapy is possible with a nanoparticle-like TLR9 agonist, which targets phagocytes in a tumor microenvironment inducing potent tumor regression.

(Materials and Methods)

The reagents, materials, animals, cells, and methods used in this Example are explained hereafter. Each Example is also supplemented with explanations when appropriate.

(Animals and Reagents)

week old female C57BL/6J mice were purchased from Nihon CLEA. Il12p40 knockout mice and Batf3 knockout mice were purchased form Jackson Laboratory. Ifnar2 knockout mice, Myd88 knockout mice, and Dectin-1 knockout mice were as previously described (Kobiyama, K., et al. Proc. Natl. Acad. Sci. U.S.A. 111, 3086-3091 (2014)). All animal experiments were conducted in accordance with the institutional guidelines of the National Institute of Biomedical Innovation. K3 was synthesized by GeneDesign. Ovoalbumin (OVA) was purchased from Seikagaku Corporation.

(Cell Strain)

EL4 and OVA expressing EL4 (EG7) is a thymoma cell line of C57BL/6J mice, which were purchased from ATCC. B16 (melanoma) was purchased from the Japanese Collection of Research Bioresourses. B16F10 (melanoma) was purchased from the RIKEN Cell Bank. MC38 (colon cancer) was provided by Dr. F. JAMES Primus. Pan02 (pancreatic cancer) was purchased from Jackson's Laboratory. EL4, EG7, MC38, and Pan02 were cultured in complete RPMI (RPMI 1640 supplemented with 10% (v/v) fetal bovine serum (FBS), penicillin, and streptomycin). B16 and B16F10 were cultured in complete DMEM (DMEM supplemented with 10% (v/v) fetal bovine serum (FBS), penicillin, and streptomycin).

(Tumor Experiment and Therapeutic Method)

The right abdomen of mice was subcutaneously (s.c.) inoculated with EG7, EL4, B16, B16F10, and MC38 cells (100 μl at 5×10⁶ cells/mL in 10% Matrigel/PBS). The tumor size was measured by the length (L), width (W), and height (H) of tumor. The tumor volume (V) was calculated as V=L×W×H. Intratumoral injection (i.t.) was directly injected into the tumorous site. CpG therapy was started after the tumor volume reached about 100 mm³. The timing thereof was 7 days after inoculation of EG7 and B16F10, 10 days after inoculation of B16, and 14 days after inoculation of MC38. Tumor carrying mice were treated three times every other day with K3 (30 μg) or K3-SPG (10 μg).

(Pan02 Peritoneal Inoculation Model)

In a Pan02 peritoneal inoculation model, 1×10⁶ Pan02 cells (100 μl at 1×10⁷ cells/mL in PBS) were injected into the abdominal cavity. CpG therapy was started 11 days after inoculation. All tumor nodules were extracted from the peritoneum of the mice on day 21. The weight thereof (g) was subsequently measured. The dosage used in the CpG therapy was as described above.

(In Vivo Imaging Experiment)

To assess the localization of K3 and K3-SPG, C57BL/6 mice were s.c. inoculated with EG7 on day 0. PBS (control), Alexa 647-K3 (30 μg) or Alexa 647-K3-SPG (10 μg) were i.v. administered on day 12. 1 hour after administration, the mice were analyzed with IVIS® Lumina Imaging System and an analysis software (Ver. 2.6, Xenogen). Images measured by relative fluorescence were converted into a unit or measurement of surface radiance (photons/sec/cm²/sr). To detect labeled CD8⁺ T cells in vivo, splenocytes were collected on day 14 from EG7 carrying C57BL/6 mice or Il12p40-Ifnar2 double knockout mice treated or untreated with K3-SPG on days 7, 9, and 11. After suspending the splenocytes, red blood cells were dissolved in ACK lysis buffer (150 mM NH₄Cl, 10 mM KHCO₃, 0.1 mM Na₂EDTA), and the cells were maintained in complete RPMI. CD8α⁺ T cells were sorted by MACS (Miltenyi Biotec). The CD8α⁺ T cells were sorted by a negative selection method. The sorted CD8α⁺ T cells were then stained with Xenolight DiR®. The stained CD8α⁺ T cells were transferred into recipient mice (C57BL/6 mice or Il12p40-Ifnar2 double knockout mice that were inoculated with EG7 on day 0 and were (or were not) i.v. treated with K3-SPG on days 7, 9, and 11) on day 14. 24 hours after transferring in stained cells, the mice were analyzed with IVIS® Lumina Imaging System (Ver. 2.6). The region of interest was consolidated to the tumor region, and the fluorescence intensity was analyzed with Living Image Software (Ver. 2.6, Xenogen).

(Immunohistochemistry)

Alexa 647-K3 (30 μg), Alexa 647-K3-SPG (10 μg), and dextran-PE (20 μg) were i.v. injected from the caudal vein into C57BL/6J mice (6 to 8 weeks old, female, CLEA Japan). Tumor was collected 1 hour after the injection. Frozen sections were immobilized for 10 minutes with 4% (w/v) paraformaldehyde and were stained with anti-CD3e antibodies and anti-CD813 antibodies together with Hoechst 33258. Pictures of the cells were taken using an Olympus IX81 system. The image data was analyzed with MetaMorph.

(Depletion Experiment)

To deplete phagocytes (dendritic cells and macrophage), clodronate liposomes or control liposomes (100 nm) (Katayama Chemical) were i.v. injected into C57BL/6 mice 5 days after EG7 inoculation. To deplete CD8⁺ T cells, 200 μg of anti-CD8α antibodies were i.v. injected into the caudal vein 6 and 13 days after EG7 inoculation.

(Analysis of Splenocytes)

Splenocytes were collected on day 14 from EG7 carrying C57BL/6 mice or Il12p40-Ifnar2 double knockout mice that were (or were not) i.v. treated with K3-SPG on days 7, 9, and 11. After preparation of the splenocytes, red blood cells were dissolved in ACK lysis buffer, and the cells were maintained in complete RPMI. The splenocytes were stained with H2K^{b} OVA tetramer (MBL), anti-CD8α antibodies (KT15), anti-TCRβ antibodies (H57-597), anti-CD62L antibodies (MEL-14), and anti-CD44 antibodies (IM7), and 7-aminoactinomycin D (7AAD). The cell count of OVA tetramer+CD44⁺CD8α⁺TCRβ⁺ was determined by flow cytometry. In other experiments, prepared splenocytes were incubated with anti-CD45 antibodies, anti-CD3e antibodies, anti-CD8α antibodies, and anti-CD11a antibodies, and were then analyzed by flow cytometry.

(Assay and Immunization of CD45 Negative Cells)

Splenocytes were collected on day 12 from EG7 carrying C57BL/6 mice or Il12p40-Ifnar2 double knockout mice that were (or were not) i.v. treated with K3-SPG on days 7, 9, and 11. After preparation of the splenocytes, red blood cells were dissolved in ACK lysis buffer, and the cells were maintained in complete RPMI. The splenocytes were stained with anti-CD45 antibodies (APC). The number of CD45⁻ cells was determined by flow cytometry. Furthermore, apoptotic cell, necrotic cell, and CD45 negative liver cell populations were stained with PI and Hoechst 33342 and then were analyzed by flow cytometry. CD45⁻ cells were than sorted with INFLUX (BD Bioscience) from K3-SPG treated, tumor carrying C57BL/6 mice.

(Vaccination Model)

C57BL/6 mice were i.v. administered with 5×10⁵ CD45⁻ cells on day −7. 7 days after the immunization, the mice were s.c. inoculated on day 0 with 5×10⁵ EG7 cells.

(Cytokine Measurement)

An ELISA kit of R&D was used to measure the mouse IL-12p40, mouse IL-13, and human IFN-γ levels.

(Statistical Analysis)

One-way analysis of variance including the Mann-Whitney U test, Student's t-test, or Bonferroni's multiple comparison test was used for statistical analysis (*p<0.05; **p<0.01; ***p<0.001). Statistical analysis was performed using GraphPad Prism software (La Jolla, Calif., USA).

### Example 1: Intravenous Injection of K3-SPG Induces Strong Tumor Growth Suppression without Adding Additional Tumor Antigen

This Example demonstrated that intravenous injection of K3-SPG induces strong suppression of tumor growth without adding any tumor antigen.

(Experiment with EG7 (OVA Expressing Mouse Thymoma Cell Line) Model)

C57BL/6 mice were inoculated on the right abdomen on day 0 with EG7 (OVA expressing mouse thymoma cell line). The mice were treated three times (7, 9, and 11 days after inoculation) with PBS and equimolar amount of K3 (30 μg) or K3-SPG (10 μg) via three different routes, i.e., subcutaneous (i.d.) administration near the base of the tail, intratumoral (i.t.) administration, or intravenous (i.v.) administration. The tumor size was measured every 2 to 3 days until day 23.

(Results)

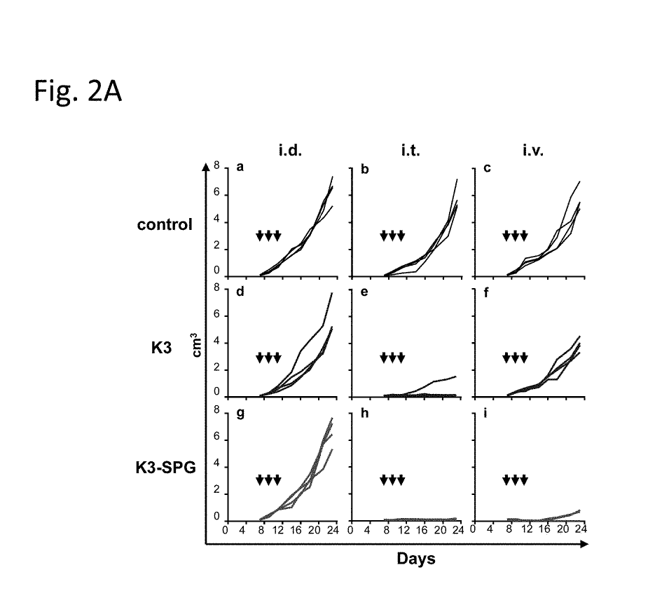

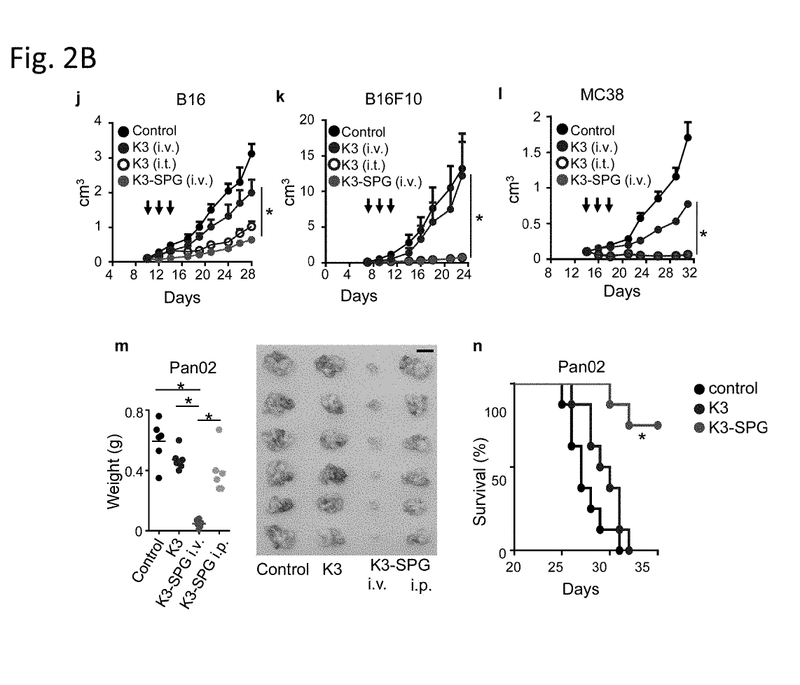

Results are shown below in FIG. 2 (A-B). In the PBS group (control), tumor growth was not suppressed via any route of administration (FIGS. 2a, b, and c (FIG. 2A)). For K3 treatment, tumor regression was observed only from i.t. but not from other routes (FIGS. 2d, e, and f (FIG. 2A)). For K3-SPG treatment, strong tumor regression was observed from both i.t. and i.v., but i.d. administration showed no effect on tumor growth (FIGS. 2g, h, and i (FIG. 2A)). Figures showing a comparison of the control, K3 and K3-SPG are shown in the Figure (FIGS. 2a, d, and g (FIG. 2A)).

Many attempts of systemic CpG ODN therapy against cancer were not successful with conventional techniques (Lou, Y., et al. Journal of immunotherapy (Hagerstown, Md.: 1997) 34, 279-288 (2011); Nierkens, S., et al. PLoS One 4, e8368 (2009)). Thus, the fact that i.v. monotherapy with K3-SPG can strongly suppress tumor growth was unexpected, demonstrating that the present invention induces an unexpected effect with respect to this point.

(Experiments with Other Tumor Cell Lines)

In order to explore the potential of such K3-SPG systemic monotherapy, other tumor cell lines were also tested under similar protocols as that used in EG7 models.

Intravenous administration of K3-SPG also suppressed the growth of melanoma (B16 and B16F10) and colon cancer (MC38) (FIGS. 2j, k, and l (FIG. 2B)). The inventors conducted further testing by making a tumor dissemination model with higher clinical malignancy. Mice were intraperitoneally (“also called i.p.”) inoculated with mouse pancreatic tumor line Pan02 (1×10⁶ cells), and K3 or K3-SPG therapy (3 times every other day) was started 11 days after the inoculation. All mice were slaughtered on day 21 to evaluate the total weight of tumor in the abdominal cavity (FIG. 2m (FIG. 2B)). Tumor growth was significantly suppressed in K3-SPG i.v. treatment group, but was not suppressed in the K3 i.p. and K3-SPG i.p. treatment groups (FIG. 2m (FIG. 2B)). Accordingly, significant survival prolongation was observed in the K3-SPG i.v. treatment group, but not in the K3 i.v. group (FIG. 2n (FIG. 2B)). These results suggested that systemic i.v. administration of K3-SPG is a promising monotherapy for many different cancers which does not require any additional tumor peptides or antigens.

### Example 2: K3-SPG Targeted Phagocytes in Tumor Microenvironment

Next, the inventors revealed the mechanism of K3-SPG in a tumor microenvironment.

K3-SPG forms nanoparticles with a size of about 30 nm (Kobiyama, K., et al. Proc. Natl. Acad. Sci. U.S.A. 111, 3086-3091 (2014)). The inventors hypotehsized that K3-SPG work through a drug delivery system to tumor (Na, J. H., et al. Journal of controlled release: official journal of the Controlled Release Society 163, 2-9 (2012); Petros, R. A. et al. Nat Rev Drug Discov 9, 615-627 (2010); Pante, N. et al. Molecular biology of the cell 13, 425-434 (2002); Davis, M. E., et al. Nat Rev Drug Discov 7, 771-782 (2008); Farokhzad, O. C. et al. ACS Nano 3, 16-20 (2009)).

(Fluorescent Label Imaging)

To test the in vivo distribution, K3 and K3-SPG were fluorescently labeled. EG7 tumor carrying mice were i.v. injected with PBS, Alexa 647-K3 (30 μg) or Alexa 647-K3-SPG (10 μg), and the distribution of fluorescence was then tested with an in vivo imaging system (IVIS).

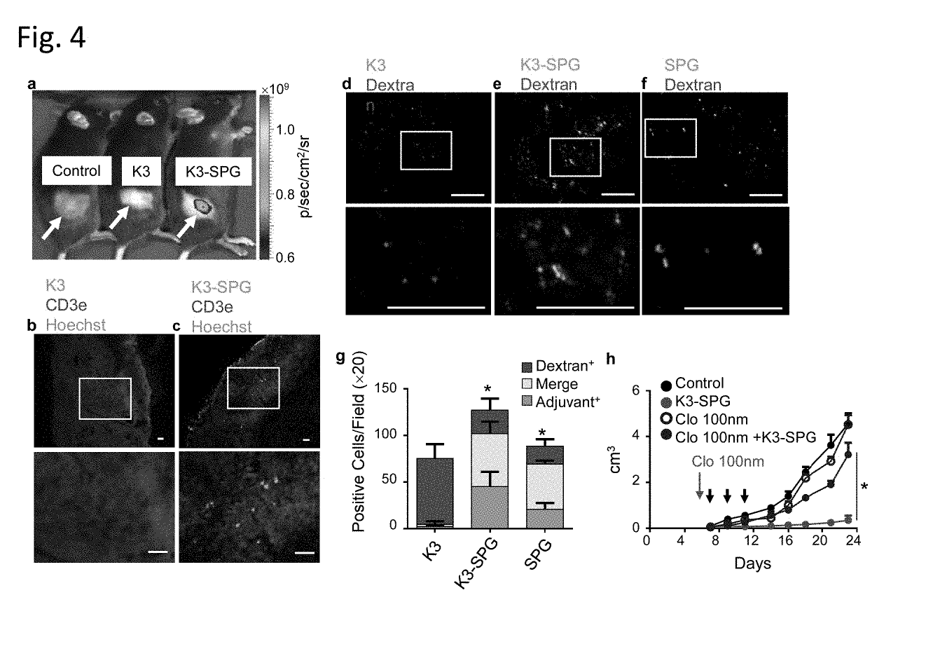

Results are shown below in FIG. 4.

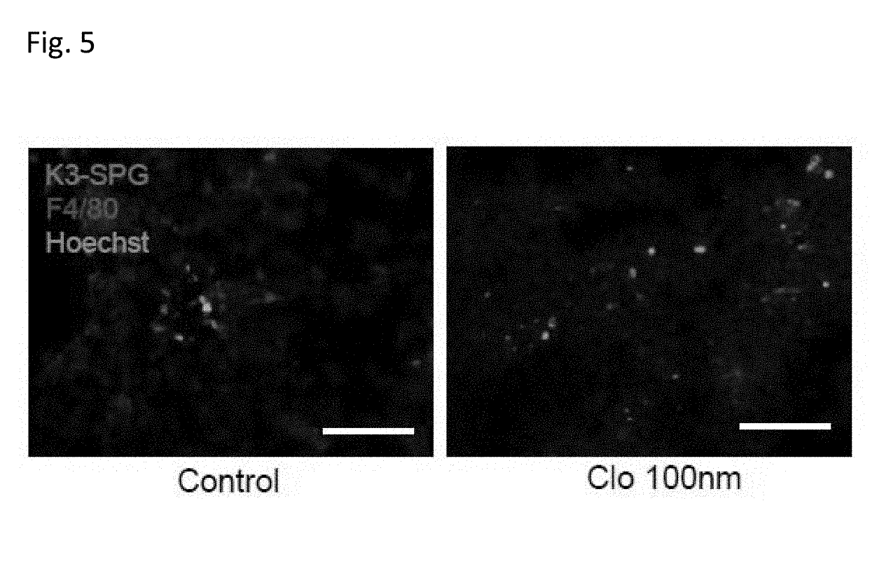

IVIS imaging revealed that K3-SPG, not K3, was accumulated at a tumor site 1 hour after i.v. administration (FIG. 4a). The accumulation of K3-SPG in the tumor appeared well-associated with tumor regression efficacy of CpG monotherapy (FIG. 2). The inventors could not detect Alexa 647-K3 in a tumor microenvironment in an immunohistochemistry (IHC) test (FIG. 4b). Meanwhile, Alexa 647-K3-SPG was found in a tumor region (FIG. 4c). The inventors could not detect any Alexa 647 signal with IHC after 24 hours. EG7 cells express CD3e on their surfaces (because EG7 is derived from a thymoma cell line), but K3-SPG was not associated with CD3e, indicating that K3-SPG was taken up by non-tumor cells. Nanoparticles were selected to be taken up by phagocytes such as macrophages and dendritic cells (DC). These cells can be labeled with TRITC-dextran in vivo. For this reason, the inventors intravenously injected TRITC-dextran with fluorescently stained K3, K3-SPG, or SPG to test the co-localization thereof by IHC (FIGS. 4d, e, and f). 1 hour after the i.v. injection, dextran was observed inside tumor regions in all samples (FIGS. 4d, e, and f), indicating that the tumor microenvironment contains phagocytes. Consistent with previous results, Alexa 647-K3 was not observed in tumor (FIG. 4d). About 50% of Alexa 647-K3-SPG and FITC-SPG observed inside tumor was co-localized with TRITC-dextran positive cells (FIGS. 4e, f, and g), indicating that K3-SPG is taken up by phagocytes in tumor microenvironment. Some of the K3-SPG were not associated with dextran. The inventors conjecture that they passively accumulated in a space inside tumor tissue via the enhanced permeability and retention (EPR) effect. To test the importance of phagocytes in K3-SPG i.v. treatment, the inventors intravenously injected clodronate liposomes. The inventors used and injected 100 nm clodronate liposomes instead of the common 200 to 300 nm liposomes to deplete the phagocytes in the tumor (Pante, N. et al. Molecular biology of the cell 13, 425-434 (2002); Pante, N. et al. Molecular biology of the cell 13, 425-434 (2002)). With this injection, most of the F4/80 positive cells in the tumor were depleted in 2 days (FIG. 5). Tumor bearing mice were or were not injected on day 5 (2 days before first K3-SPG treatment) with clodronate liposomes. Mice were treated with K3-SPG as in FIG. 2 (A-B). When clodronate liposomes were injected in advance, suppression of K3-SPG mediated tumor growth was significantly offset (p<0.05) (FIG. 4h), whereas clodronate liposome injection in and of itself did not affect tumor growth relative to PBS treated mice. These results indicate that K3-SPG targets phagocytes in a tumor microenvironment, and the anti-tumor effect of K3-SPG is mostly depended on the K3-SPG incorporation into the phagocytes in the tumor microenvironment.

### Example 3: Production of Both IL12 and IFN-I Cytokines in Tumor Microenvironment is Critical for K3-SPG Monotherapy

In this Example, the inventors tested agents that are considered necessary for the success of K3-SPG monotherapy.

Cytokines such as IL-12 and IFN-I are demonstrated to be important immunostimulatory agents for CpG ODNs (Krieg, A. M., et al. Journal of immunology 161, 2428-2434 (1998); Klinman, D. M., et al. Immunity 11, 123-129 (1999); Ishii, K. J., et al. Current opinion in molecular therapeutics 6, 166-174 (2004)) including K3-SPG (Kobiyama, K., et al. Proc. Natl. Acad. Sci. U.S.A. 111, 3086-3091 (2014)). Thus, the inventors tested whether IL-12 and IFN-I are required for tumor regression with K3-SPG therapy.

Il12p40 and IFNAR2 knockout mice were subcutaneously inoculated with EG7 cells on day 0, and were i.v. treated three times with PBS or K3-SPG (10 μg) as in FIG. 2 (A-B). The effect of K3-SPG on tumor regression was then observed.

(Results)

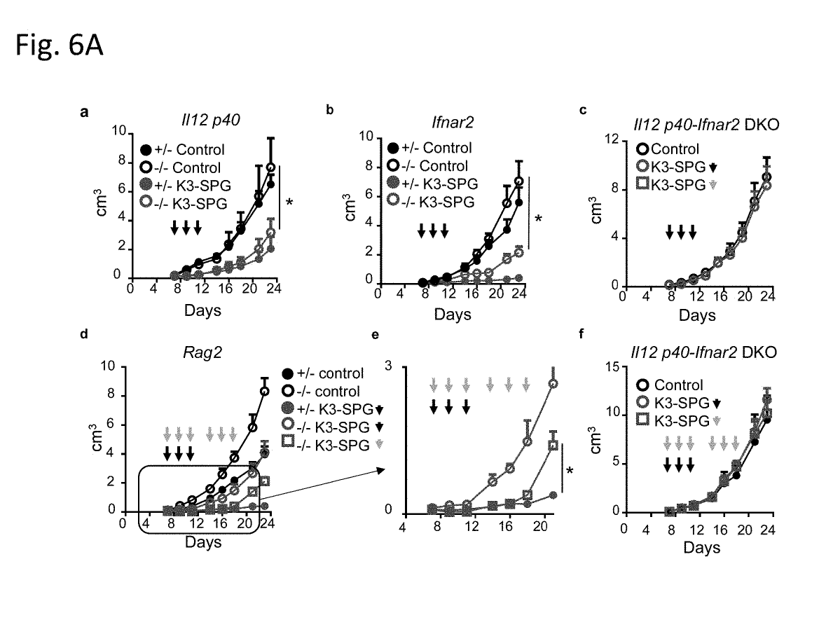

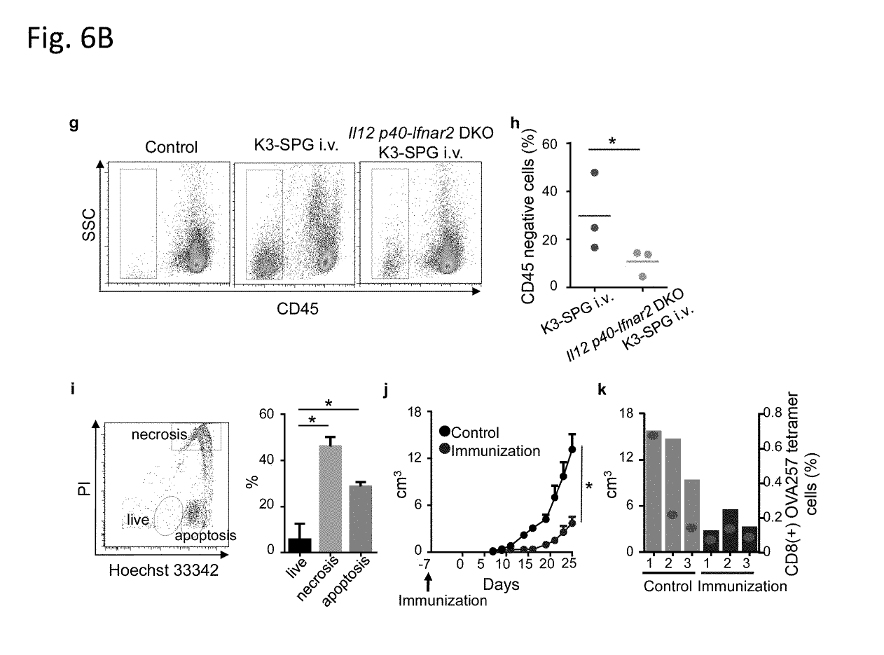

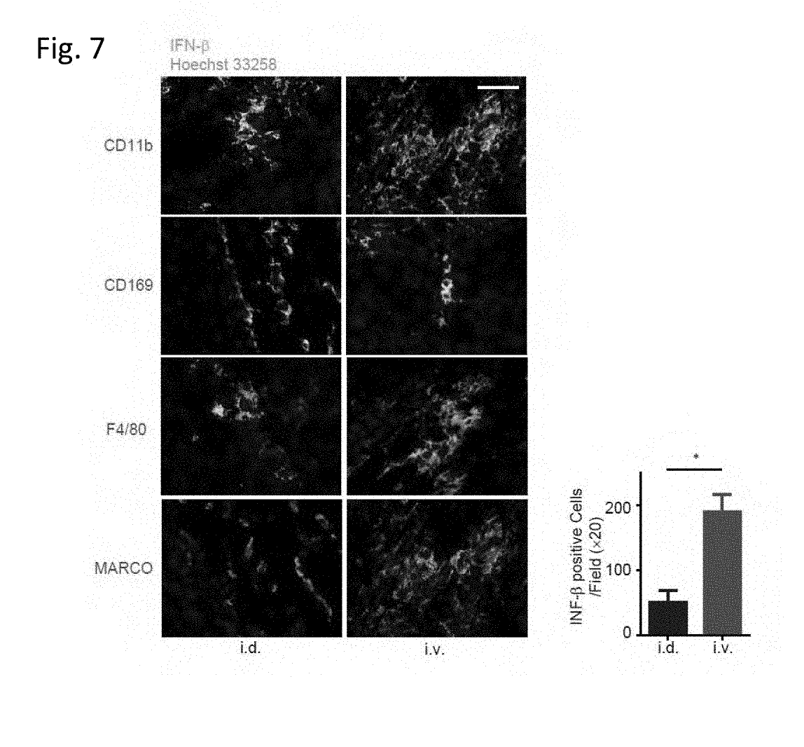

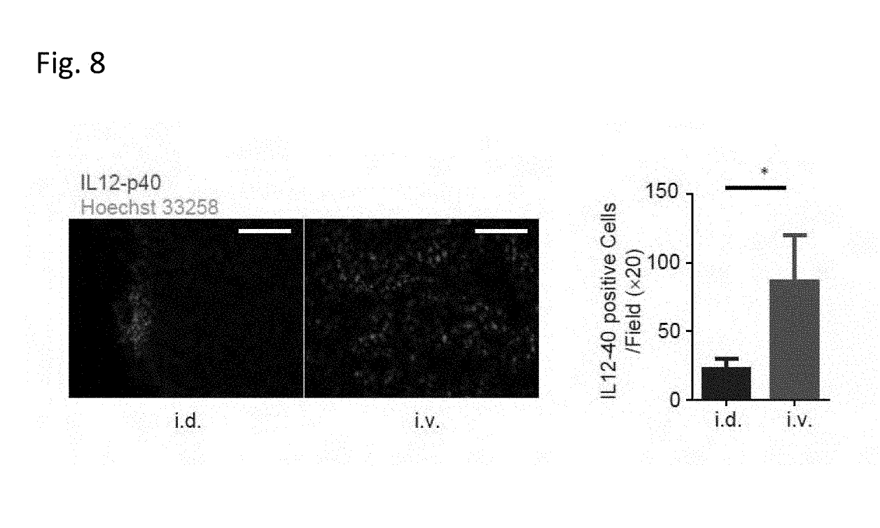

Results are shown below in FIG. 6 (A-B). The effect of K3-SPG on tumor regression was partially dependent on IL-12p40 and IFN-I signaling (FIGS. 6a and b (FIG. 6A)). The inventors also tested IL12p40 and IFNAR2 double knockout (DKO) mice to discover that the effect of K3-SPG was completely suppressed in the DKO mice (FIG. 6c (FIG. 6A)). IFN-p and IL-12p40 were also detected in tumor by IHC staining (FIGS. 7 and 8). These data show that secretion of both IL12p40 and IFN-I cytokines in tumor is critical for K3-SPG mediated tumor suppression.

The inventors also tested Rag2 mice, which were completely lacking T cell and B cell mediated adaptive immune responses. While Rag2 mice could not control all tumor growth even with K3-SPG treatment (FIG. 6d (FIG. 6A)), the inventors found that rag2 knockout mice could partially control tumor growth during three K3-SPG treatments (FIG. 6f (FIG. 6A)). To confirm this observation, the inventors prepared a group of rag2 mice treated 6 times (days 7, 9, and 11, and days 14, 16, and 18) to find that tumor was clearly controlled by this protocol in the rag2 mice (FIG. 6f (FIG. 6A)). Interestingly, IL12p40 and IFNAR2 DKO mice were completely unresponsive to K3-SPG monotherapy with this extensive treatment protocol (FIG. 6e (FIG. 6A)). These data show that K3-SPG therapy induced both IL-12p40 and IFN-I in tumor, resulting in both innate immune responses and adaptive immune response against the tumor.

### Example 4: K3-SPG Treatment Induces Tumor Cell Death in a Manner Dependent on Both IL12p40 and IFN-I

This Example demonstrated that K3-SPG treatment induces tumor cell death in a manner dependent on both IL12p40 and IFN-I.

Partial suppression of tumor growth without adaptive immunity observed in rag2 mice and complete suppression thereof in IL12p40 and IFNAR2 DKO mice were observed. Thus, the inventors tested a wide range of tumor-host interaction during K3-SPG treatment.

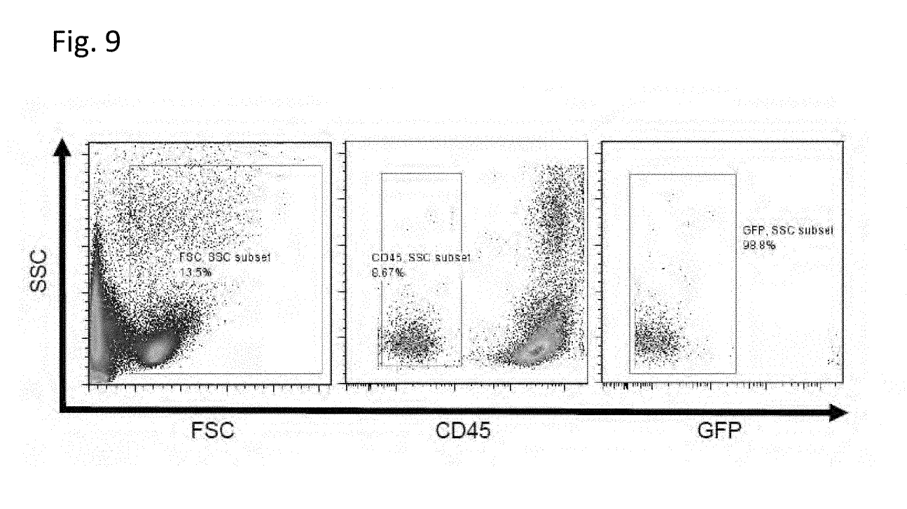

The inventors discovered that the spleen removed on day 12 (day after three treatments with K3-SPG) contained a greater amount of CD45 negative cells relative to PBS treated spleens (FIG. 6g (FIG. 6B)). Interestingly, these CD45 negative cells significantly decreased in IL12p40 and IFNAR2 DKO mice (FIGS. 6g and h (FIG. 6B)). The inventors sorted these CD45 negative cells. The size and morphology strongly indicated that the cells were derived from tumor cells. These CD45 negative cells were further confirmed to be GFP negative by an EG7 inoculation test in GFP mice, indicating that these cells were derived from tumor cells (FIG. 9). Since EG7 cells do not express CD45, CD45 being negative also supports the hypothesis. With Hoechst and PI staining, most of the CD45 negative cells in the spleen were dead cells with both apoptotic and necrotic characteristics (FIG. 6i (FIG. 6B)). These data show that tumor phagocytes targeted by K3-SPG secreted IL-12p40 and IFN-I in the tumor microenvironment, and these cytokines induced tumor cell death and the cells were released into circulation and finally trapped in the spleen.

### Example 5: Released Dead Tumor Cells Induce Antitumor CTLs Against Multiple Tumor Antigens

This Example demonstrated that released dead tumor cells induce antitumor CTLs against multiple tumor antigens.

To test the immunogenicity of these CD45 negative cells found in the spleen of K3-SPG treated mice, the inventors sorted the cells and intravenously injected the cells into naïve mice as immunization. EG7 tumor cells were then transplanted into the immunized mice 7 days after administration of the sorted cells. The CD45 negative cell immunized mice significantly protected against EG7 tumor growth (FIG. 6j (FIG. 6B)). Interestingly, OVA257 tetramer positive cells in the control mice and immunized mice (red dots in FIG. 6k (FIG. 6B)) did not correlate with tumor size (bars in FIG. 6k (FIG. 6B)), indicating that immunization by CD45 negative cells induced more effective immune responses against EG7 tumor than only the OVA257 epitope (FIG. 6k (FIG. 6B)). These results show that K3-SPG monotherapy induces tumor cell death which is dependent on both IL-12 and IFN-I and the dead tumor cells function as an effective immunogen for antitumor immune responses.

(CD8 T Cells are Important Effectors in K3-SPG Mediated Tumor Regression)

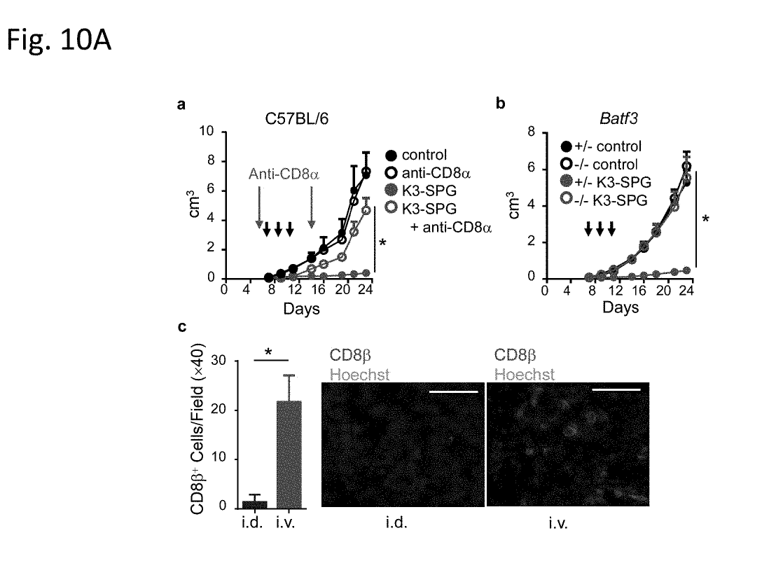

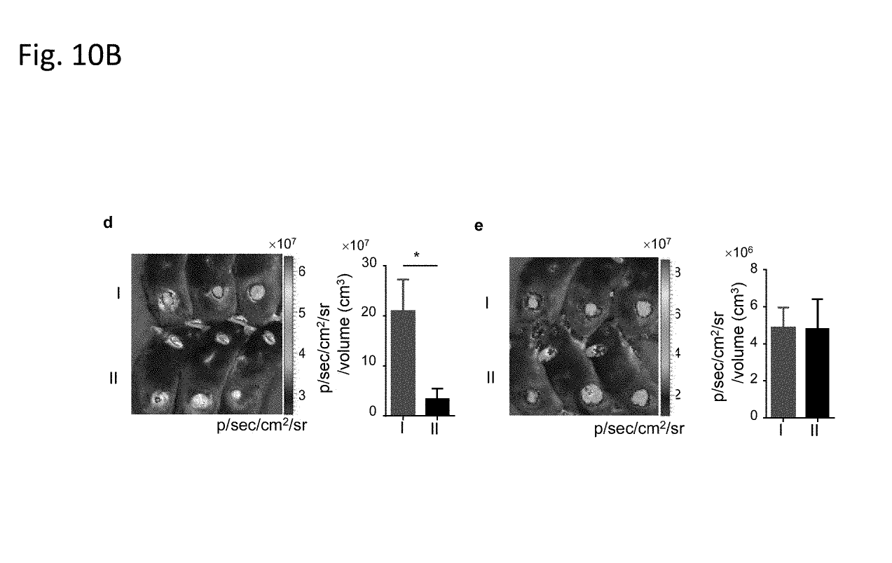

Results with Rag2 mice indicated that a tumor suppressing effect of K3-SPG was also dependent on adaptive immune responses. Thus, the inventors tested CD8 T cells required for K3-SPG therapy. Depletion of CD8 T cells in vivo significantly suppressed the antitumor effect of K3-SPG (FIG. 10a (FIG. 10A)), indicating that CD8 T cells are important effector cells in the K3-SPG therapy. Tumor regression with K3-SPG was also dependent on Batf3 (lacking cross-presenting CD8α⁺ DC) (FIG. 10b (FIG. 10A)), indicating that K3-SPG monotherapy also enhanced CD8α⁺ DC mediated cross-presentation. The inventors observed a clear association between CD8 T cell tumor growth and tumor infiltration. CD8 T cells accumulated in a tumor region in the K3-SPG i.v. group, but not in the i.d. group (FIG. 10c (FIG. 10A)).

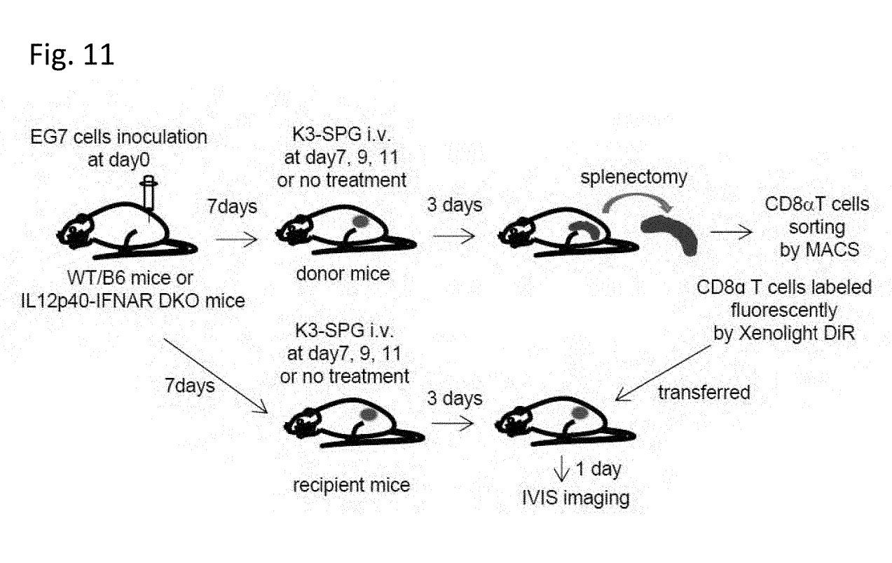

Finally, the inventors tested the requirement for these CD8 T cells to enter the tumor region. WT mice and Il12p40-Ifnar2 DKO mice were inoculated on day 0 with EG7 cells, and were i.v. treated with K3-SPG or PBS on days 7, 9, and 11. On day 14, CD8α⁺ cells were purified from the spleens of these mice, stained with Xenolight DiR®, and transferred into other EG7 carrying mice treated with K3-SPG (days 7, 9, and 11) (14 days after inoculation). The distribution of Xenolight DiR® labeled CD8 T cells were then analyzed on day 15 with IVIS (FIG. 11). On day 15, CD8 T cells derived from donor mice carrying untreated tumor did not accumulate at a tumor site of WT recipient mice, even when treated with K3-SPG (FIG. 10d (FIG. 10B), II). Meanwhile, CD8 T cells derived from K3-SPG treated tumor carrying donor mice were detected at a tumor site of recipient mice (FIG. 10d (FIG. 10B), I), indicating that K3-SPG monotherapy induced antitumor CD8 T cells that can migrate to and infiltrate a tumor microenvironment. These in vivo activated CD8 T cells were able to enter the tumor microenvironment of DKO recipient mice (FIG. 10e (FIG. 10B)). Even if IL-12 and IFN-I were important in the induction of CD8 T cells and innate immunity with systemic K3-SPG monotherapy, the results show that, once CD8 T cells are activated with K3-SPG therapy, secretion of IL-12 and IFN-I cytokines in the tumor microenvironment is not necessarily required for CD8 T cell tumor infiltration. In summary, these results demonstrated that tumor specific CD8 T cell activation is sufficient for tumor infiltration. Surprisingly, infiltration of these CD8 T cells is not dependent on cytokine production in the tumor microenvironment.

### Discussion

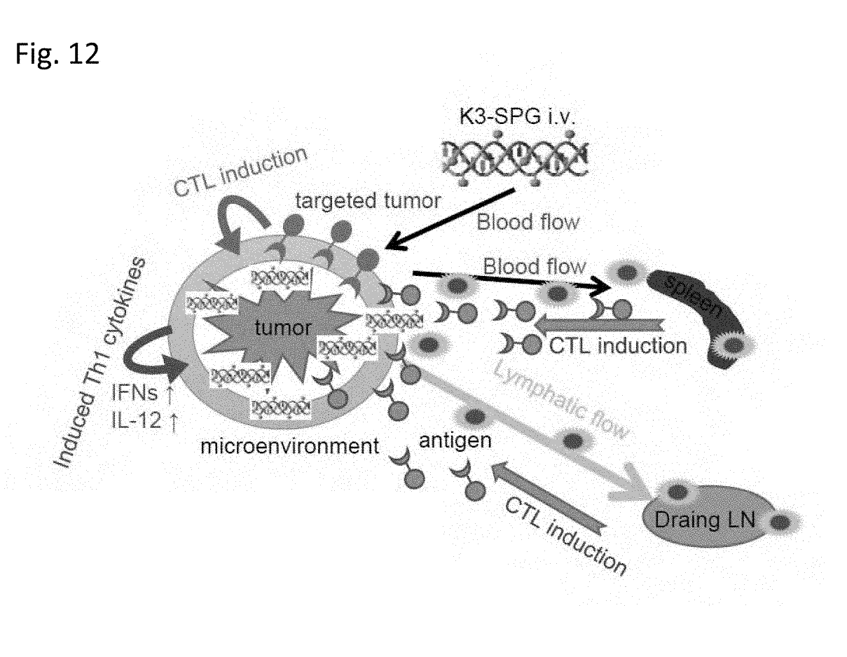

The inventors showed the possibility of novel cancer immunotherapy. This is a novel therapy, in which CpG is targeted to phagocytes in a tumor microenvironment (FIG. 12). CpG induces an immune response by immune cells via stimulation of TLR9 to activate macrophages and DCs in particular (Klinman, D. M., et al. Immunity 11, 123-129 (1999); Ishii, K. J., et al. Current opinion in molecular therapeutics 6, 166-174 (2004)). Such activation is very important for anticancer immune responses. In previous reports, CpG had to be administered directly into tumor. However, a DDS function is added to a complex of SPG and CpG, and efficacy that is equivalent or greater than intratumoral administration is exhibited with systemic administration (Schettini, J., et al. Cancer immunology, immunotherapy: CII 61, 2055-2065 (2012); Lou, Y., et al. Journal of immunotherapy (Hagerstown, Md.: 1997) 34, 279-288 (2011); Nierkens, S., et al. PLoS One 4, e8368 (2009); Heckelsmiller, K., et al. Journal of immunology 169, 3892-3899 (2002); Ishii, K. J., et al. Current opinion in molecular therapeutics 6, 166-174 (2004)), such that the inventors solved this problem. A complex of SPG and CpG by nanoparticle formation (Kobiyama, K., et al. Proc. Natl. Acad. Sci. U.S.A. 111, 3086-3091 (2014)) was able to be stabilized in vivo. It was found that this effect can target the tumor environment, so that TLR9 immunocompetent cells were subjected to the tumor environment. This novel CpG developed by the inventors is engulfed by phagocytes to form nanoparticles.

Subsequently, the phagocytes that have engulfed the novel CpG produce cytokines such as IFN and IL-12 in the tumor environment. Induction of these cytokines in the tumor environment is very important. Previous reports describe that IFNβ therapy directly targeting the tumor environment make dendritic cells migrate into the tumor and increase antigen cross-presentation in the tumor microenvironment to reactivate CTL. These cytokines induce cell death of tumor cells. Furthermore, the inventors discovered that this effect is exerted by activation of innate immunity. The cell death plays a very important role. This was a liaison between innate and adaptive immunity. Acquired immunity is induced by releasing tumor cell death from the tumor microenvironment. The immunogenic tumor cell death induces multiple cytotoxic T lymphocytes. Tumor specifically induced CTLs in vivo as described above can infiltrate the tumor microenvironment in response to tumor. This antitumor immunity system can use endogenous antigens to cope with immunoediting that is a barrier for cancer immunotherapy.

Circulation of tumor cells after K3-SPG monotherapy can function as a biomarker with excellent treatment effect on tumor.

### Example 6: Formulation Example

For example, formulation was prepared as follows.

7.22 mg of K3-dA₄₀ (SEQ ID NO: 2) was dissolved in water (3.7 mL), and SPG (15 mg) was dissolved in 0.25N NaOH (1 mL). 330 mM NaH2PO4 with a volume of 1 mL was added to a DNA solution, and the SPG solution was then added to the DNA/NaH₂PO₄ solution. The mixture was maintained overnight at 4° C. to complete the complex formation. The formulation can be manufactured by fixing the mole ratio (M_{SPG}/M_{DNA}) to 0.27.

The agents used in the formulation are available from GeneDesign, invivogen, Wako, or the like.

As described above, the present invention is exemplified by the use of its preferred embodiments. However, it is understood that the scope of the present invention should be interpreted solely based on the Claims. It is also understood that any patent, any patent application, and any references cited herein should be incorporated herein by reference in the same manner as the contents are specifically described herein.

### INDUSTRIAL APPLICABILITY

The present invention provides a novel form of anticancer agent that can be used as a monotherapy. Thus, the complex of the invention is useful in the pharmaceutical field as an anticancer agent.

### [Sequence Listing Free Text]

### SEQ ID NO: 1: K3

### SEQ ID NO: 2: K3-dA₄₀

SEQ ID NO: 3: dA₄₀-K3

### SEQ ID NO: 4: K3-dA20

### SEQ ID NO: 5: K3-dA25

### SEQ ID NO: 6: K3-dA30

### SEQ ID NO: 7: K3-dA35

1. An anticancer agent comprising a complex, comprising:
(a) an oligodeoxynucleotide comprising a humanized K type CpG oligodeoxynucleotide and polydeoxyadenylic acid, wherein the polydeoxyadenylic acid is disposed on the 3′ side of the humanized K type CpG oligodeoxynucleotide; and
(b) β-1,3-glucan.

2. A method for treating cancer in a subject comprising the step of administering the anticancer agent of claim 1 to the subject without a cancer antigen.

3. A method for treating cancer in a subject comprising the step of administering the anticancer agent of claim 1, to a reticuloendothelial system and/or a lymph node in the subject.

4. The method of claim 3, wherein the reticuloendothelial system and/or lymph node comprises tumor and phagocytes.

5. The method of claim 3, wherein the reticuloendothelial system comprises a spleen and/or a liver.

6. The method of claim 3, wherein the anticancer agent is administered without a cancer antigen.

7. The method of claim 2, wherein the administration comprises systemic administration.

8. The method of claim 7, wherein the systemic administration is selected from intravenous administration, intraperitoneal administration, oral administration, subcutaneous administration, intramuscular administration, or intratumoral administration.

9. The anticancer agent of claim 1, wherein the oligodeoxynucleotide is selected from the group consisting of K3 (SEQ ID NO: 1), K3-dA₄₀ (SEQ ID NO: 2), dA₄₀-K3 (SEQ ID NO: 3), K3-dA20 (SEQ ID NO: 4), K3-dA25 (SEQ ID NO: 5), K3-dA30 (SEQ ID NO: 6), and K3-dA35 (SEQ ID NO: 7).

10. The anticancer agent of claim 1, wherein the β-1,3-glucan is selected from the group consisting of schizophyllan (SPG), lentinan, scleroglucan, curdlan, pachyman, grifolan, and laminaran.

11. The anticancer agent of claim 1, wherein the complex is K3-SPG.

12. A method for inducing accumulation of dead cancer cells in a reticuloendothelial system and/or a lymph node in a subject, comprising the step of administering a complex to the subject, wherein the complex comprises:
(a) an oligodeoxynucleotide comprising a humanized K type CpG oligodeoxynucleotide and polydeoxyadenylic acid, wherein the polydeoxyadenylic acid is disposed on the 3′ side of the humanized K type CpG oligodeoxynucleotide; and
(b) β-1,3-glucan.

13. The method of claim 12, wherein the oligodeoxynucleotide is selected from the group consisting of K3 (SEQ ID NO: 1), K3-dA₄₀ (SEQ ID NO: 2), dA₄₀-K3 (SEQ ID NO: 3), K3-dA20 (SEQ ID NO: 4), K3-dA25 (SEQ ID NO: 5), K3-dA30 (SEQ ID NO: 6), and K3-dA35 (SEQ ID NO: 7).

14. The method of claim 12, wherein the β-1,3-glucan is selected from the group consisting of schizophyllan (SPG), lentinan, scleroglucan, curdlan, pachyman, grifolan, and laminaran.

15. The method of claim 12, wherein the complex is K3-SPG.

16. The method of claim 12, wherein the reticuloendothelial system and/or lymph node comprises tumor and phagocytes.

17. The method of claim 12, wherein the reticuloendothelial system comprises a spleen and/or a liver.

18. The method of claim 12, wherein the administration comprises systemic administration.

19. The method of claim 18, wherein the systemic administration is selected from intravenous administration, intraperitoneal administration, oral administration, subcutaneous administration, intramuscular administration, or intratumoral administration.

20. A method for the expression of interleukin 12 (IL12) and/or interferon (IFN) γ or the enhancement thereof, comprising the step of administering a composition to the subject, wherein the composition comprises:
(a) an oligodeoxynucleotide comprising a humanized K type CpG oligodeoxynucleotide and polydeoxyadenylic acid, wherein the polydeoxyadenylic acid is disposed on the 3′ side of the humanized K type CpG oligodeoxynucleotide; and
(b) β-1,3-glucan.

21. The method of claim 20, wherein the oligodeoxynucleotide is K3 (SEQ ID NO: 1), K3-dA₄₀ (SEQ ID NO: 2), dA₄₀-K3 (SEQ ID NO: 3), K3-dA20 (SEQ ID NO: 4), K3-dA25 (SEQ ID NO: 5), K3-dA30 (SEQ ID NO: 6), and K3-dA35 (SEQ ID NO: 7).

22. The method of claim 20, wherein the β-1,3-glucan is selected from the group consisting of schizophyllan (SPG), lentinan, scleroglucan, curdlan, pachyman, grifolan, and laminaran.

23. The method of claim 20, wherein the complex is K3-SPG.

24. The method of claim 3, wherein the administration comprises systemic administration.

25. The method of claim 24, wherein the systemic administration is selected from intravenous administration, intraperitoneal administration, oral administration, subcutaneous administration, intramuscular administration, or intratumoral administration.

26. The method of claim 13, wherein the β-1,3-glucan is selected from the group consisting of schizophyllan (SPG), lentinan, scleroglucan, curdlan, pachyman, grifolan, and laminaran.

27. The method of claim 21, wherein the β-1,3-glucan is selected from the group consisting of schizophyllan (SPG), lentinan, scleroglucan, curdlan, pachyman, grifolan, and laminaran.
